SEQUENCE LISTING (20 sequences)

SEQ ID NO: 1 - DNA, 21 bp, Artificial Sequence
  Description of Artificial Sequence Synthetic primer
gctcttactgactggcatgag

SEQ ID NO: 2 - DNA, 20 bp, Artificial Sequence
  Description of Artificial Sequence Synthetic primer
cgcagctctaggagcatgtg

SEQ ID NO: 3 - DNA, 20 bp, Artificial Sequence
  Description of Artificial Sequence Synthetic primer
ccaagaggtgagtgcttccc

SEQ ID NO: 4 - DNA, 22 bp, Artificial Sequence
  Description of Artificial Sequence Synthetic primer
ctgttgttcagactctctccct

SEQ ID NO: 5 - DNA, 21 bp, Artificial Sequence
  Description of Artificial Sequence Synthetic primer
cagctgggctgtacaaacctt

SEQ ID NO: 6 - DNA, 21 bp, Artificial Sequence
  Description of Artificial Sequence Synthetic primer
cattggaagtgaagcgtttcg

SEQ ID NO: 7 - DNA, 22 bp, Artificial Sequence
  Description of Artificial Sequence Synthetic primer
aggagctgtcattagggacatc

SEQ ID NO: 8 - DNA, 22 bp, Artificial Sequence
  Description of Artificial Sequence Synthetic primer
ctccaagccaaagtccttagag

SEQ ID NO: 9 - DNA, 21 bp, Artificial Sequence
  Description of Artificial Sequence Synthetic primer
ctctgttcagctattggacgc

SEQ ID NO: 10 - DNA, 23 bp, Artificial Sequence
  Description of Artificial Sequence Synthetic primer
cggaatttctgggattcagcttc

SEQ ID NO: 11 - DNA, 22 bp, Artificial Sequence
  Description of Artificial Sequence Synthetic primer
tcagggagtaatggttggaatg

SEQ ID NO: 12 - DNA, 22 bp, Artificial Sequence
  Description of Artificial Sequence Synthetic primer
ggcagtagtcacgaaggaatag

SEQ ID NO: 13 - DNA, 21 bp, Artificial Sequence
  Description of Artificial Sequence Synthetic primer
ggagaaacctgccaagtatga

SEQ ID NO: 14 - DNA, 20 bp, Artificial Sequence
  Description of Artificial Sequence Synthetic primer
cctgttgctgtagccgtatt

SEQ ID NO: 15 - DNA, 22 bp, Artificial Sequence
  Description of Artificial Sequence Synthetic primer
cccaacttcttagagggacaag

SEQ ID NO: 16 - DNA, 20 bp, Artificial Sequence
  Description of Artificial Sequence Synthetic primer
gcttatgacccgcacttact

SEQ ID NO: 17 - DNA, 22 bp, Artificial Sequence
  Description of Artificial Sequence Synthetic primer
aaacaaggagattttcctccgc

SEQ ID NO: 18 - DNA, 21 bp, Artificial Sequence
  Description of Artificial Sequence Synthetic primer
gtccagggcatctgaagcatt

SEQ ID NO: 19 - DNA, 22 bp, Artificial Sequence
  Description of Artificial Sequence Synthetic primer
caggtctggcaattcttctgaa

SEQ ID NO: 20 - DNA, 23 bp, Artificial Sequence
  Description of Artificial Sequence Synthetic primer
gtcttgctcatgtgtgtaagtga

(19) United States
(12) Patent Application Publication
(10) Pub. No.: US 2019/0105260 A1
(43) Pub. Date: Apr. 11, 2019

(54) COMPOSITIONS AND METHODS TO PROMOTE WOUND HEALING

(71) Applicant: The Regents of the University of California, Oakland, CA (US)

(72) Inventors: Roslyn Rivkah Isseroff, Davis, CA (US); Danielle Marie Tartar, Davis, CA (US); Chuong Minh Nguyen, Davis, CA (US); Farzam Gorouhi, Davis, CA (US)

(21) Appl. No.: 16/087,941

(22) PCT Filed: Mar. 21, 2017
(86) PCT No.: PCT/US2017/023428

§ 371 Date: Sep. 24, 2018

Related U.S. Application Data
(60) Provisional application No. 62/311,516, filed on Mar. 22, 2016.

(51) Int. Cl. A61K 9/00; A61K 31/138; A61K 31/5377; A61P 25/24; A61K 35/28; A61L 27/36; A61L 27/54; A61L 27/24; A61K 9/70

(52) U.S. Cl. CPC A61K 9/0014; A61K 31/138; A61K 31/5377; A61P 25/24; A61K 35/28; A61K 9/7023; A61L 27/3633; A61L 27/3604; A61L 27/54; A61L 27/24; A61L 27/3687

(57) ABSTRACT

Provided are compositions and methods for the treatment of epithelial wound, particularly chronic or non-healing wounds.

### CROSS-REFERENCE TO RELATED APPLICATIONS

This application claims the benefit under 35 U.S.C. § 119(e) of U.S. Provisional Application No. 62/311,516, filed on Mar. 22, 2016, which is hereby incorporated herein by reference in its entirety for all purposes.

### BACKGROUND

Chronic non-healing wounds place an increasing burden on healthcare systems. Most of these wounds can be ascribed to one of three etiologies: diabetic ulcers, venous ulcers or pressure ulcers. In the case of diabetic ulcers, it is estimated that 40-60% of diabetes (DB) patients are at risk for the development of DB foot complications, and DB foot wounds (DFW) account for over 20% of all hospitalizations of DB patients. These slow healing or non-healing wounds are extremely unmanageable resulting in an estimated 82,000 non-traumatic lower limb amputations each year, in other words, one amputation every 30 seconds in DB patients. Chronic inflammation is a hallmark of diabetic and other types of non-healing wounds. Stress plays an important role in impairing wound healing and sustaining chronic wound inflammation. Some of the most potent mediators of stress are the catecholamines, such as epinephrine and norepinephrine. It has been shown that epinephrine can activate beta adrenergic receptors on human keratinocytes and mesenchymal stem cells causing them to release inflammatory mediators such as IL-6, thus contributing to the prolonged inflammation

Current methods for treating DFW includes debridement to remove necrotic and infected tissues, dressings to provide a moist wound environment, and topical applications of antimicrobial or biologic agents, offloading, physical therapies, and educational strategies. However, these different treatment modalities often fail to achieve complete wound closure since they do not address the main culprit, i.e., persistent inflammation. For example, excessive use of antibiotics may address bacterial numbers and to some extent inflammation but can lead to the development of resistant strains.

### SUMMARY

In one aspect, provided are pharmaceutical compositions. In varying embodiments, the compositions comprise comprising as sole pharmacologically active agent (e.g., does not comprise a second pharmacologically active agent) an antidepressant in a pharmaceutically acceptable carrier, wherein the composition is formulated for topical delivery of the antidepressant to a tissue or organ. In varying embodiments, the compositions comprise as sole first and second pharmacologically active agents (e.g., does not comprise a third pharmacologically active agent) an antidepressant in combination with a beta adrenergic receptor antagonist, both active agents in a pharmaceutically acceptable carrier, wherein the composition is formulated for topical delivery of the antidepressant and the beta adrenergic receptor antagonist to a tissue or organ. In varying embodiments, the tissue or organ is other than the eye (e.g., the composition is not an ophthalmic formulation). In varying embodiments, the tissue or organ comprises an epithelial tissue, e.g., skin. In varying embodiments, the composition is formulated for topical delivery of the antidepressant, optionally in combination with the beta adrenergic receptor antagonist, to skin. In some embodiments, the antidepressant increases extracellular serotonin levels. In varying embodiments, the antidepressant is selected from the group consisting of a selective serotonin reuptake inhibitor (S SRI), a serotonin-norepinephrine reuptake inhibitor (SNRI), a tricyclic or tetracyclic antidepressant (TCA), a monoamine oxidase inhibitor (MAOI) and an atypical antidepressant. In varying embodiments, the selective serotonin reuptake inhibitor (SSRI) is selected from the group consisting of fluoxetine, citalopram, escitalopram, fluvoxamine, fluvoxamine CR, paroxetine, paroxetine CR, and sertraline. In varying embodiments, the serotonin-norepinephrine reuptake inhibitor (SNRI) is selected from the group consisting of desvenlafaxine, duloxetine, venlafaxine, venlafaxine XR, milnacipran, and levomilnacipran. In varying embodiments, the beta adrenergic receptor antagonist is a non-selective antagonist for β1 and β2 adrenergic receptors. In varying embodiments, the beta adrenergic receptor antagonist is selected from carteolol, carvedilol, labetalol, nadolol, penbutolol, pindolol, propranolol, sotalol, timolol, and mixtures, analogs and salts thereof. In varying embodiments, the beta adrenergic receptor antagonist is a selective antagonist for β1 adrenergic receptors. In varying embodiments, the beta adrenergic receptor antagonist is selected from acebutolol, atenolol, betaxolol, bisoprolol, celiprolol, esmolol, metoprolol, nebivolol, and mixtures, analogs and salts thereof. In varying embodiments, the beta adrenergic receptor antagonist is a selective antagonist for β2 adrenergic receptors. In varying embodiments, the β2 adrenergic receptor antagonist is selected from butoxamine and ICI-118,551. In varying embodiments, the beta adrenergic receptor antagonist is selected from the group consisting of timolol, labetalol, dilevelol, propanolol, carvedilol, nadolol, carteolol, penbutolol, sotalol, ICI-118,551, butoxamine, and mixtures, analogs and salts thereof. In varying embodiments, the beta adrenergic receptor antagonist is substantially free of activity as a beta-3 adrenergic receptor agonist. In varying embodiments, the composition comprises the antidepressant as sole pharmacologically active agent or the antidepressant in combination with a beta adrenergic receptor antagonist at a concentration in the range of about 0.001% w/v to about 30% w/v, e.g., from about 0.001% w/v to about 0.2% w/v, 0.5% w/v, 1.0% w/v, 2.0% w/v, 5.0% w/v, 10.0% w/v, 15.0% w/v, 20.0% w/v, 25.0% w/v, or 30.0% w/v. In some embodiments, the composition comprises one or both of the antidepressant and the beta adrenergic receptor antagonist in a subtherapeutic dose. In varying embodiments, the composition further comprises mesenchymal stem cells (MSCs). In some embodiments, the MSCs have been have been contacted and/or pre-conditioned with an antidepressant (e.g., SSRI, SNRI) and/or beta adrenergic receptor antagonist; the embodiments of the pharmacologically active agents as described above and herein). In some embodiments, the MSCs have been cultured in medium comprising the antidepressant and/or beta adrenergic receptor antagonist. In some embodiments, the MSCs have been cultured at least 24 hours in medium comprising the antidepressant and/or beta adrenergic receptor antagonist. In some embodiments, the MSCs have been cultured under hypoxic conditions. In varying embodiments, the MSCs are derived from a tissue selected from the group consisting of adipose, bone marrow, dermis, placenta, umbilical cord, and Wharton's jelly. In varying embodiments, the MSCs are human. In varying embodiments, the MSCs are autologous to the subject. In varying embodiments, the MSCs are allogeneic to the subject. In varying embodiments, the composition comprises a gel, liquid, ointment, cream, lotion, suspension, spray or foam.

In a further aspect, provided is an extracellular matrix scaffold comprising the composition, as described above and herein. In varying embodiments, the extracellular matrix scaffold comprises collagen. In varying embodiments, the extracellular matrix scaffold comprises collagen-glycosaminoglycan biodegradable matrix.

In a further aspect, provided are wound dressings. In varying embodiments the wound dressings comprise the compositions and/or the extracellular matrix scaffolds, as described above and herein. In varying embodiments, the wound dressings are impregnated with the composition and/or extracellular matrix scaffold. In varying embodiments, at least one surface of the dressing is coated with the composition and/or extracellular matrix scaffold.

In a further aspect, provided are kits. In varying embodiments, the kits comprise the compositions and/or the extracellular matrix scaffolds and/or wound dressings, as described above and herein.

In a further aspect, provided are methods for increasing a rate of wound healing in a subject in need thereof. In varying embodiments, the methods comprise:

a) identifying a subject suffering from a wound in an epithelial tissue; and

b) topically administering the composition and/or extracellular matrix scaffold and/or wound dressing, as described above and herein, to the subject. In some embodiments, the epithelial tissue comprises skin. In some embodiments, an antidepressant is administered as the sole pharmacologically active agent in a therapeutically effective dose. In some embodiments, a combination of an antidepressant and a beta adrenergic receptor antagonist are topically co-administered and one or both of the antidepressant and the beta adrenergic receptor antagonist are administered in a therapeutically effective dose. In some embodiments, a combination of an antidepressant and a beta adrenergic receptor antagonist are topically co-administered and one or both of the antidepressant and the beta adrenergic receptor antagonist are administered at a subtherapeutic dose. The embodiments of the pharmacologically active agents are as described above and herein. In some embodiments, the composition and/or extracellular matrix scaffold and/or wound dressing comprises MSCs, and the MSCs are autologous, syngeneic, allogeneic or xenogeneic to the subject. In some embodiments, the wound comprises a chronic skin wound. In varying embodiments, the wound is secondary to vascular insufficiency/injury (e.g., diabetic neuropathy, digital ischemia, Raynaud's phenomenon) or a connective tissue disease (e.g., scleroderma, vasospasm). In some embodiments, the wound comprises a venous stasis ulcer, a diabetic foot ulcer, a peripheral digit ulcer, a neuropathic ulcer, or a decubitus ulcer. In some embodiments, the wound comprises a wound resulting from surgical wound dehiscence. In some embodiments, the wound comprises an incision, laceration, abrasion, or ulcer. In some embodiments, the wound comprises a burn. In some embodiments, the epithelial tissue comprises a genitourinary epithelium, a gastrointestinal epithelium, a pulmonary epithelium, or a corneal epithelium. In some embodiments, the rate of wound healing is at least about 5% greater, e.g., at least about 10%, 15%, 20%, 25% greater or more, in comparison to an untreated individual or the same subject prior to topical administration of the composition. In some embodiments, the subject is a human, a non-human primate, a canine, a feline, an equine, a bovine, an ovine or a porcine. In varying embodiments, the antidepressant or the combination of the antidepressant and the beta adrenergic receptor antagonist is administered to the subject multiple times. In varying embodiments, the antidepressant or the combination of the antidepressant and the beta adrenergic receptor antagonist is administered to the subject at least once daily. In varying embodiments, the antidepressant or the combination of the antidepressant and the beta adrenergic receptor antagonist is administered to the subject at least once daily for at least 10 days, e.g., for at least 15, 20, 30 days or more, or until the wound is substantially or completely healed.

### Definitions

Unless defined otherwise, all technical and scientific terms used herein have the same meaning as commonly understood by one of ordinary skill in the art. The following definitions supplement those in the art and are directed to the current application and are not to be imputed to any related or unrelated case, e.g., to any commonly owned patent or application. Although any methods and materials similar or equivalent to those described herein can be used in the practice for testing, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a receptor” includes a plurality of receptors; reference to “a cell” includes mixtures of cells, and the like.

The term “about” as used herein indicates the value of a given quantity varies by +/−10% of the value, or optionally +/−5% of the value, or in some embodiments, by +/−1% of the value so described.

The term “topical” refers to administration or delivery of a compound (e.g., a beta adrenergic receptor antagonist) by application of the compound to a surface of a body part. For example, a compound can be topically administered by applying it to skin, to the surface of a wound within the skin, a mucus membrane, or wound within the mucous membrane, or another body surface, or wound within. Topical administration can result, e.g., in either local or systemic delivery of a compound.

An “antagonist” is a compound (e.g., a drug) that can bind to a receptor and prevent an agonist from binding to and activating that receptor. Typically, binding of an antagonist to a receptor forms a complex which does not give rise to any response, as if the receptor were unoccupied. Alternatively, the antagonist can be a partial agonist.

It is worth noting that certain compounds can be classified as both an agonist and an antagonist. For example, a “mixed agonist-antagonist” (also called a “partial agonist”) is a compound which possesses affinity for a receptor, but which, unlike a full agonist, will elicit only a small degree of the response characteristic of that receptor, even if a high proportion of receptors are occupied by the compound. Such occupancy of the receptors by the partial agonist can prevent binding of a full agonist (e.g., an endogenous agonist) to the receptor.

The term “co-administering” or “concurrent administration”, when used, for example with respect to the compounds (e.g., one or more antagonists of a beta-adrenergic receptor) and/or analogs thereof and another active agent (e.g., an anesthetic, an antibiotic), refers to administration of the compound and/or analogs and the active agent such that both are in the blood at the same time. Co-administration can be concurrent or sequential.

The term “therapeutically effective amount” refers to that amount of the compound being administered sufficient to prevent or decrease the development of one or more of the symptoms of the disease, condition or disorder being treated. When administered as the sole pharmacologically active agent, the antidepressant is generally administered in a therapeutically effective amount.

The terms “prophylactically effective amount” and “amount that is effective to prevent” refer to that amount of drug that will prevent or reduce the risk of occurrence of the biological or medical event that is sought to be prevented. In many instances, the prophylactically effective amount is the same as the therapeutically effective amount.

“Subtherapeutic dose” refers to a dose of a pharmacologically active agent(s), either as an administered dose of pharmacologically active agent, or actual level of pharmacologically active agent in a subject that functionally is insufficient to elicit the intended pharmacological effect in itself (e.g., to promote wound healing), or that quantitatively is less than the established therapeutic dose for that particular pharmacological agent (e.g., as published in a reference consulted by a person of skill, for example, doses for a pharmacological agent published in the Physicians' Desk Reference, 70th Ed., 2016, PDR Network, or Brunton, et al., Goodman & Gilman's The Pharmacological Basis of Therapeutics, 12th edition, 2011, McGraw-Hill Education/Medical). A “subtherapeutic dose” can be defined in relative terms (i.e., as a percentage amount (less than 100%) of the amount of pharmacologically active agent conventionally administered). For example, a subtherapeutic dose amount can be about 1% to about 75% of the amount of pharmacologically active agent conventionally administered. In some embodiments, a subtherapeutic dose can be about 75%, 50%, 30%, 25%, 20%, 10% or less, than the amount of pharmacologically active agent conventionally administered. When co-administered with a beta adrenergic receptor antagonist and/or mesenchymal stem cells, one or both of the antidepressant and the beta adrenergic receptor antagonist can be administered in a subtherapeutic dose.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies (e.g., epithelial and/or cutaneous wound healing, closure, re-epithelialization and/or dermal regeneration), or one or more symptoms of such disease or condition.

As used herein, the phrase “consisting essentially of” refers to the genera or species of active pharmaceutical agents recited in a method or composition, and further can include other agents that, on their own do not have substantial activity for the recited indication or purpose.

The terms “subject,” “individual,” and “patient” interchangeably refer to any mammal, including humans and non-human mammals, e.g., primates, domesticated mammals (e.g., canines and felines), agricultural mammals (e.g., bovines, ovines, equines, porcines) and laboratory mammals (e.g., rats, mice, rabbits, guinea pigs, hamsters), as described herein.

The terms “increasing,” “promoting,” “enhancing” with respect to wound healing refers to increasing the epithelialization, closure and/or dermal regeneration of a wound in a subject by a measurable amount using any method known in the art. The wound healing is increased, promoted or enhanced if the re-epithelialization, closure and/or dermal regeneration of the wound is at least about 10%, 20%, 30%, 50%, 80%, or 100% increased in comparison to the re-epithelialization, closure and/or dermal regeneration of the wound prior to administration of beta adrenergic receptor antagonist conditioned mesenchymal stem cells (MSCs), e.g., over a predetermined time period. In some embodiments, the re-epithelialization, closure and/or dermal regeneration of the wound is increased, promoted or enhanced by at least about 1-fold, 2-fold, 3-fold, 4-fold, or more in comparison to the re-epithelialization, closure and/or dermal regeneration of the wound prior to administration of the beta adrenergic receptor antagonist conditioned MSCs.

The terms “reducing,” “decreasing” with respect to wound size refers to reducing or decreasing the open wound surface area or the wound volume in a subject by a measurable amount using any method known in the art. The wound surface area or volume in a subject is reduced or decreased if the measurable parameter of the wound is at least about 10%, 20%, 30%, 50%, 80%, or 100% reduced or decreased in comparison to the measurable parameter of the one or more symptoms prior to administration of the beta adrenergic receptor antagonist conditioned MSCs. In some embodiments, the measurable parameter of the wound surface area or volume is reduced or decreased by at least about 1-fold, 2-fold, 3-fold, 4-fold, or more in comparison to the measurable parameter of the one or more symptoms prior to administration of the beta adrenergic receptor antagonist conditioned MSCs.

The term “mesenchymal stem cells” refers to stem cells defined by their capacity to differentiate into bone, cartilage, and adipose tissue. With respect to cell surface markers, MSCs generally express CD44 and CD90, and should not express CD34, CD45, CD80, CD86 or

The term “chronic wound” refers to wounds that do not heal completely after receiving standard medical treatment for 30 days” (as defined by US Center for Medicare and Medicaid Services (Cms.gov, 2005)).

### BRIEF DESCRIPTION OF THE DRAWINGS

FIGS. 1A-C illustrate that Bone Marrow-Derived MSC (BMSC) produce serotonin (5-HT), and serotonin increases BMSC proliferation and migration. Panel A: ELISA data demonstrating serotonin production by five different primary BMSC lines. Panel B: Migration assay demonstrating that serotonin itself increases migration of BMSC which aids the recruitment of MSC to wound sites. Panel C: Serotonin increases proliferation of BMSC in vitro.

FIGS. 2A-C illustrate that serotonin (5-HT) increases keratinocyte migratory speed and re-epithelialization in a scratch wound ex vivo model, likely through the ERK-STAT3-NFkB pathway. Panel A: Single cell migration assay with a human keratinocyte cell line, HaCaT, demonstrating that 5-HT increases keratinocyte migration in vitro. Panel B: Scratch wound assays at 6-hour time point confirmed the enhanced migration of primary keratinocytes from 3 different donors by serotonin in a dose-dependent manner which suggests enhanced in vivo re-epithelization rate. Panel C: Multiplex data showing that addition of serotonin significantly increases phosphorylated ERK, phosphorylated STAT3 and NF-kB in keratinocytes.

FIGS. 3A-B illustrate that MSC (both adipose- and bone marrow-derived), serotonin, and fluoxetine all inhibit T cell proliferation in vitro, independent of serotonin receptor 5HTR2A. Panel A: In a mixed lymphocyte reaction, 250,000 Carboxyfluorescein succinimidyl ester (CFSE)-labeled CD4+CD25-effector T cells were cultured with 100,000 CD11c+ dendritic cells as antigen. 3,000 AMSC, 1,500 BMSC, 1 μM fluoxetine or 10 μM serotonin was additionally added to co-cultures. Proliferation was measured based on CFSE dilution. Panel B: Average of either 4 (T+AMSC, T+AMSC+DC) or 2 replicates (T cells alone, T+DC, T+BMSC, T+DC+BMSC, T+DC+FLX, T+DC+5-HT, T+DC+FLX+5HTR2Ai, T+DC+5-HT+5HTR2Ai), where 5HTR2Ai=ketanserin. P values were derived using a 2-tailed t-test.

FIGS. 4A-D illustrate topical fluoxetine and topical serotonin improve wound healing in a diabetic mouse model of delayed wound healing. Panel A: respective images of wounds treated with either vehicle alone (polyethylene glycol, or PEG), 0.2% fluoxetine (FLX), 2% FLX, 0.2% serotonin (5-HT), or 2% 5-HT. Panel B: Histomorphometric analysis of wounds. Representative histology sections for each group were chosen. Following euthanasia of animals, wounds were excised, biopsied and bisected. Wound tissue was fixed in paraformaldehyde for 12-16 hours and then paraffin embedded. Serial 5 μm sections were obtained from the center of the wound and the section with the largest wound bed (as defined by distance between hair follicles) was stained with hemotoxylin and eosin. Images were taken using 10× objective on a Keyance microscope. Panels C-D: Re-epithelialization was determined on sections created as in panel B. Image J analysis software was utilized to divide the re-epithelialized tissue (distance from hair follicle to remaining wound) by the entire original wound surface (as defined as distance between hair follicles). Panel D provides the distribution of data depicted in Panel C. P values were derived using a 2-tailed t-test.

FIG. 5 illustrates ELISA data demonstrating that nearly half of serotonin in culture is degraded within 24 hours, illustrating our choice for daily topical delivery.

FIGS. 6A-C illustrate that topical fluoxetine and topical serotonin decreased neutrophil infiltration while increasing macrophage number and wound bed vascularity. 13 week-old Db/Db female mice underwent full-thickness excisional biopsies with 8 mm punch biopsies. Wounds were splinted to prevent contraction. At day 10, animals were sacrificed and wounds were harvested to assess re-epithelialization. Representative histology sections for each group were chosen. Following euthanasia of animals, wounds were excised, biopsied and bisected. Wound tissue was fixed in paraformaldehyde for 12-16 hours and then paraffin embedded. Serial 5 μM sections were obtained from the center of the wound and the section with the largest wound bed (as defined by distance between subcutaneous fat tissue—indicated by yellow arrows in figure). Images were taken using 10× objective on a Keyance microscope. Sections were stained with antibody to Gr-1 (A), CD31 (B) or F4/80 (C) to assess neutrophil, endothelial cell or macrophage numbers, respectively, in the wound beds. Wound bed area was outlined in Spectrum software (demarcated by yellow arrows in this figure), which then calculated the number of positive cells. Data is presented as positive cells/mm². P values were derived using a 1-tailed t-test. *, p<0.05; **, p<0.01. N=5/group.

FIGS. 7A-E illustrate the effects of topical serotonin and fluoxetine on the local wound bed cytokine milieu. 13 week-old Db/Db female mice underwent full-thickness excisional wounding with 8 mm punch biopsy tool. Wounds were splinted to prevent contraction. At day 10, animals were sacrificed and RNA was harvested from wound beds. RT-PCR was performed for the genes shown and data was first normalized to geometric means of housekeeping genes (GAPDH, HSP90, 18S ribosomal RNA). A. TGF-β; B. IL-1β; C. TNF-α; D. IL-10; E. α-smooth muscle actin (α-SMA). Fold change of fluoxetine (FLX) or serotonin (5HT)-treated animals is shown compared to vehicle (polyethylene glycol) control by the delta-delta Ct method. Data represented as average±SEM.

FIGS. 8A-D illustrate that fluoxetine enhances macrophage polarization towards an anti-inflammatory M2 phenotype. Bone marrow described macrophages from wild-type C57/B6J mice were harvested and cultured under polarizing conditions towards M0 (no polarization; normal culture medium), M1 (using LPS and IFNγ) or M2 (using IL-4 and IL-13). Fluoxetine (100 nM) was added to test wells. M2 activation status was assessed using RT-PCR (normalized to HSP90) for CD206, Ym-1 and Arg-1. M1 activation status was assessed using TNFα (A, B). C: At day 7 of macrophage maturation, adherent bone marrow derived monocytes in culture were identified with macrophage markers (CD11b and F4/80) using flow cytometry. After 24 hours, CD206 expression was identified in treated group (C). Results were then quantified and reported (D). Data presented as average±SEM. ** p<0.01.

FIG. 9 illustrates that serotonin increases wound healing in vitro and fluoxetine further enhances these effects through 5HTR1A and 5HTR2A. Neonatal Human Keratinocytes (NHKs) were either non-treated (Control) or treated with 5-HT (1 μM) and FLX (0.01 μM, 0.1 μM, 1 μM) and/or 5HTR1A blocker (100 nM NAN190) and/or 5HTR2A antagonist (1 μM Ketanserin or KET). NHKs from at least 3 different donors were plated at fully confluent density and treated with Mitomycin to prevent proliferation. P-200 tips were used to induce a scratch wound in the middle of each well and time-lapse images were recorded every 30 minutes for 6 hours and % healed were calculated as previously described. Data represented as Mean±SEM. Student t-tests were used for statistical analysis. *, p<0.05; **, p<0.01; ***, p<0.001.

FIGS. 10A-B illustrate that combinatorial therapy of topical fluoxetine and topical timolol increased re-epithelialization in a diabetic mouse model of impaired wound healing. 13 week-old Db/Db female mice underwent full-thickness excisional biopsies with 8 mm punch biopsies. Wounds were splinted to prevent contraction. At day 12, animals were sacrificed and wounds were harvested to assess re-epithelialization. Representative histology sections for each group were chosen. Following euthanasia of animals, wounds were excised, biopsied and bisected. Wound tissue was fixed in paraformaldehyde for 12-16 hours and then paraffin embedded. Serial 5 μM sections were obtained from the center of the wound and the section with the largest wound bed (as defined by distance between subcutaneous fat tissue) was stained with hemotoxylin and eosin. Images were taken using 10× objective on a Keyance microscope. (A) Re-epithelialization was determined on sections created. Image J analysis software was utilized to divide the re-epithelialized tissue by the entire original wound surface. (B) Percent of wounds at different stages of re-epithelialization was quantified. Data represented as Mean±SEM. P values were derived using a 2-tailed t-test. N=5/group.

FIG. 11 illustrates that combinatorial therapy of topical fluoxetine and fluoxetine-pretreated MSC increased re-epithelialization in a diabetic mouse model of impaired wound healing. 13 week-old Db/Db female mice underwent full-thickness excisional wounding with 8 mm punch biopsy tool. All wounds were splinted to prevent contraction. Wounds were then treated with INTEGRA™ alone, INTEGRA™ plus 1 μM Fluoxetine, INTEGRA™ plus MSC (250,000 cells) or INTEGRA™ plus 1 μM Fluoxetine plus Fluoxetine-pretreated MSC (250,000 cells). At day 11, animals were sacrificed and wounds were harvested to assess re-epithelialization. Representative histology sections for each group were chosen. Following euthanasia of animals, wounds were excised, biopsied and bisected. Wound tissue was fixed in paraformaldehyde for 12-16 hours and then paraffin embedded. Serial 5 μM sections were obtained from the center of the wound and the section with the largest wound bed (as defined by distance between subcutaneous fat tissue) was stained with hemotoxylin and eosin. Images were taken using 10× objective on a Keyance microscope. Re-epithelialization was determined on sections created. Image J analysis software was utilized to divide the re-epithelialized tissue by the entire original wound surface. Data represented as Mean±SEM. N=5/group.

### DETAILED DESCRIPTION

### 1. Introduction

Mesenchymal stem cells (MSC) represent a recently-utilized tool in the therapy of chronic diabetic ulcers. We provide data herein demonstrating that MSC aid in wound healing by producing serotonin. Furthermore, topical preparations comprising an antidepressant (e.g., a selective serotonin reuptake inhibitor (SSRI), or a serotonin-norepinephrine reuptake inhibitor (SNRI)) find use to promote, accelerate and/or facilitate the healing of chronic wounds, and can optionally be used in combination with a topically administered beta adrenergic receptor antagonist and/or MSC therapy.

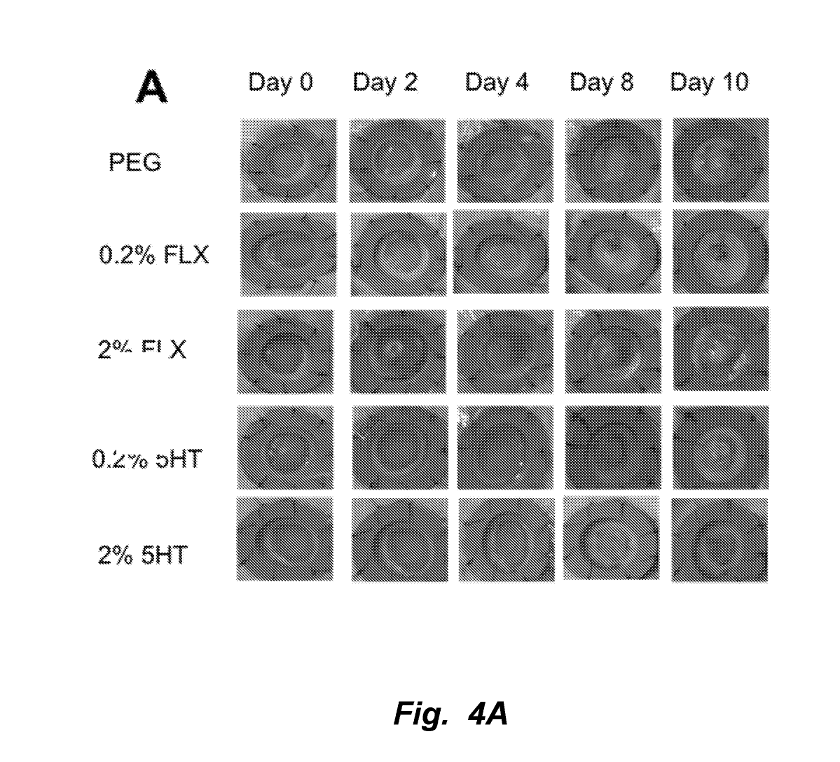

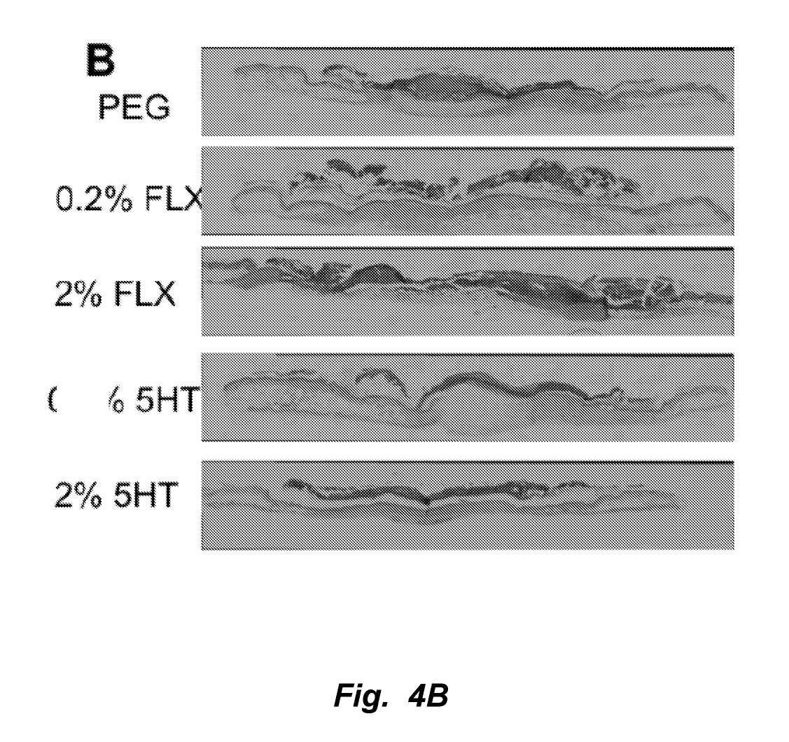

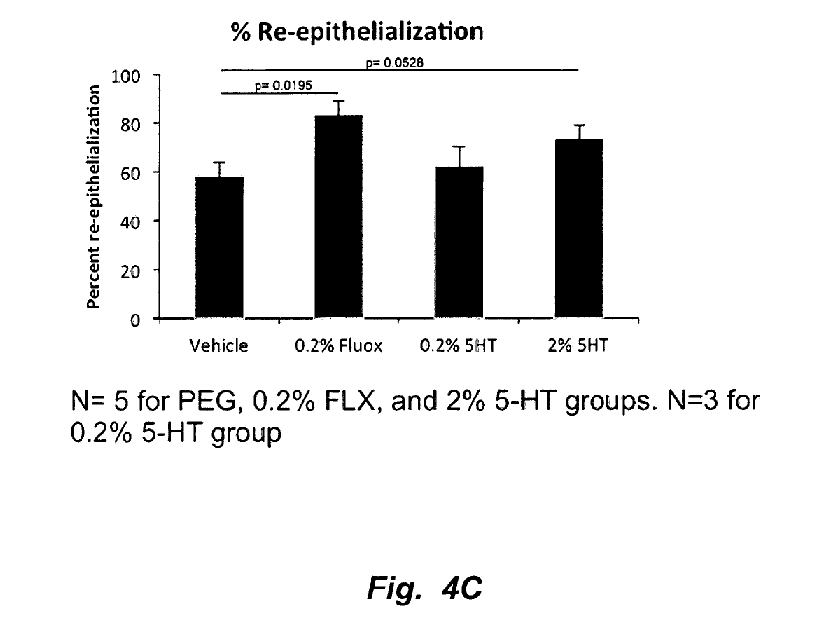

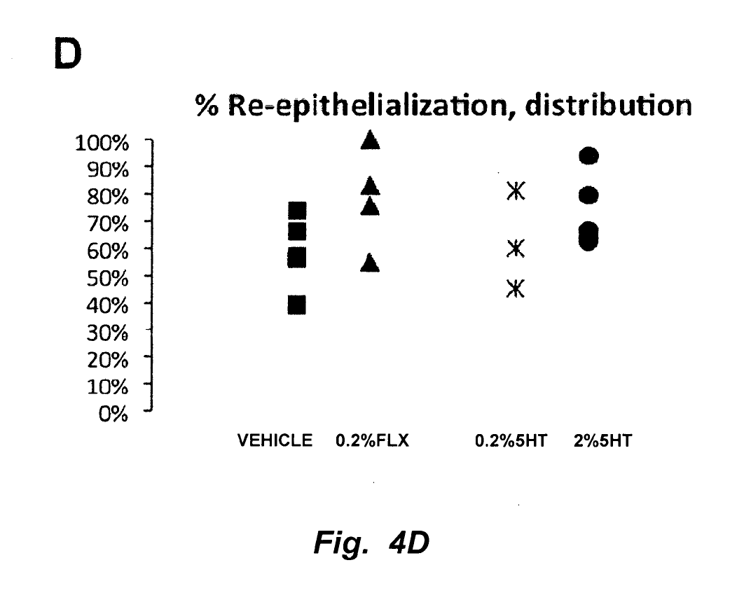

Serotonin produced by MSCs increases MSC proliferation and more importantly, keratinocyte proliferation and migration. Keratinocytes are the skin cells required to fill in wounded tissue. Additionally, MSCs exert anti-inflammatory effects in vitro and in vivo. This is important because chronic wounds are stuck in the “inflammatory” phase of wound healing. As demonstrated herein, topical serotonin and topical fluoxetine (an illustrative SSRI) improve wound healing in a diabetic model of impaired wound healing. See, FIG. 4.

Further provided are methods for the use mesenchymal stem cells (MSC) embedded in an extracellular matrix Scaffold for Dermal Regeneration (SDR), e.g., for the treatment of epithelial and/or cutaneous wounds, e.g., diabetic wounds, venous and decubitus ulcers. In certain embodiments, the MSCs have been preconditioned by exposure to an antidepressant (e.g., an SSRI and/or SNRI) or a beta adrenergic receptor antagonist.

The present methods, compositions and kits are based, in part, on the surprising discovery that the topical administration of an antidepressant (e.g., an SSRI and/or an SNRI) as sole pharmacologically active agent, or combined with one or more of a beta adrenergic receptor antagonist, MSCs, and SDR is therapeutically effective for the treatment of epithelial and/or cutaneous wounds, e.g., diabetic wounds. We have shown previously that stress-induced inflammatory responses in full thickness cutaneous wounds of diabetic mice can be reduced by the addition of beta adrenergic receptor antagonists. See, e.g., U.S. Patent Publ. No. 2010/0215710 and WO 2015/031110, both of which are hereby incorporated herein by reference in their entireties for all purposes. Current topical therapies focus on the use of medications such as antibiotics, hyperbaric oxygen, all-trans retinoic acid, antimicrobials, among other treatments, but no treatments have utilized topically formulated antidepressants (e.g., an SSRI and/or SNRI) administered as sole pharmacologically active agent or in combination with one or more of a beta adrenergic receptor antagonist, MSCs, and SDR.

We have shown previously that inflammation plays a pivotal role in impaired wound healing in diabetic mice. Furthermore, we have shown that epinephrine can increase the release of inflammatory mediators and that use of a beta adrenergic receptor antagonist, such as timolol or ICI-118551, leads to suppression of the release of IL-6 by human keratinocytes. MSCs are useful cellular therapy candidates for wound healing and commercially available extracellular matrix scaffolds for dermal regeneration (e.g., INTEGRA™) are currently in use for the treatment of burn and other wounds. Herein we demonstrate that antidepressant and/or beta adrenergic receptor antagonist-preconditioned MSCs in SDR are useful for the suppression of the inflammatory response in a cutaneous wound and improve healing of the wound. Further, a topical solution of an antidepressant as sole pharmacologically active agent, or in combination with a beta adrenergic receptor antagonist (e.g., timolol), can be applied to the MSC+SDR implanted, sutured or embedded in the cutaneous wound (e.g., every other day or as appropriate) to continue the conditioning of the MSC with the antidepressant alone or in combination with the beta adrenergic receptor antagonist.

Beta adrenergic receptor antagonists are widely used in medical practice both as systemic agents for cardiovascular disease and as topical agents for the eye. Illustrative beta adrenergic receptor antagonists of use include without limitation timolol and ICI-118551. Timolol is already in use as an FDA approved drug for use for glaucoma. INTEGRA™ (SDR) is an FDA approved wound care device comprised of a porous matrix of cross-linked bovine tendon collagen and glycosaminoglycan. The collagen-glycosaminoglycan biodegradable matrix provides a scaffold for MSC retention. Preconditioning of MSC+SDR with an antidepressant, alone or in combination with a beta adrenergic receptor antagonist promotes and facilitates embedded or transplanted MSC survival and function better in the catecholamine rich wound microenvironment.

Current topical methods for treating DFW includes debridement to remove necrotic and infected tissues, dressings to provide a moist wound environment, bandages, and topical applications of antimicrobial or biologic agents, offloading, physical therapies, and educational strategies. However, these different treatment modalities often fail to achieve complete wound closure since they do not address the main culprit, i.e., persistent inflammation. For example, excessive use of antibiotics may address bacterial numbers and to some extent inflammation but can lead to the development of resistant strains. The present methods, compositions and kits address the inflammatory response without promoting or causing undesirable side effects like the development of antibiotic-resistant bacteria.

### 2. Conditions Subject to Treatment

Subjects who can benefit generally have an epithelial and/or cutaneous wound. A wound in an epithelial tissue typically disrupts the continuity of the epithelial layer. For example, a wound in the skin typically disrupts (e.g., completely removes a section of) the epidermis, and, depending on the depth of the wound, can also remove part of the dermis. Healing of a wound in an epithelial tissue generally involves migration and/or proliferation of cells surrounding the wound, and the wound is typically considered to be healed when the wound is re-epithelialized, e.g., covered by at least one layer of cells.

In one aspect, the present compositions, extracellular matrix scaffolds and methods provide for increasing the rate of repair, re-epithelialization and dermal regeneration of wounds in epithelial tissues, e.g., in humans. In varying embodiments, the methods entail contacting a wound with a composition comprising an antidepressant as sole pharmacologically active agent or in combination with a beta adrenergic receptor antagonist, wherein the composition is formulated for topical delivery. In varying embodiments, the methods involve the suturing, embedding and/or implanting of an extracellular matrix comprising embedded mesenchymal stem cells that have been exposed to, pre-conditioned with and/or cultured in the presence of an antidepressant (e.g., SSRI and/or SNRI) as sole pharmacologically active agent or in combination with a beta adrenergic receptor antagonist to stimulate, promote and/or facilitate wound repair (e.g., re-epithelialization of the area), e.g., by stimulating migration and/or proliferation of epithelial cells (e.g., of keratinocytes for repair of a wound in the skin) and by decreasing the mediators of wound inflammation.

In varying embodiments, the target patient is a subject comprising or at risk for comprising a wound in an epithelial tissue. In varying embodiments, the wound is in skin. The methods and matrices described herein can be particularly useful for stimulating healing of chronic, non-healing skin wounds, which oftentimes are not sterile. In varying embodiments, the wound is secondary to a systemic illness, e.g., diabetes, peripheral ischemia secondary to vascular insufficiency/injury or vasospasm (e.g., Raynaud's phenomenon), a connective tissue disorder (e.g., scleroderma). In some embodiments, the wound comprises a chronic skin wound, e.g., a venous stasis ulcer, a diabetic foot ulcer, a peripheral digital ulcer, a neuropathic ulcer, or a decubitus ulcer. Other exemplary chronic wounds for which the methods can be used include, but are not limited to, other chronic ulcers such as immune-mediated (e.g., rheumatoid arthritis or inflammatory bowel disease-related) ulcers, radiotherapy-induced ulcers, and ulcers resulting from vasculitis, arteriolar obstruction or occlusion, pyoderma gangrenosum, thalessemia, and other dermatologic diseases that result in non-healing wounds. In a related class of embodiments, the wound results from surgical wound dehiscence.

The methods and matrices described herein can also be applied to other types of wounds. For example, the wound can comprise a burn, cut, incision, laceration, ulceration, abrasion, or essentially any other wound in an epithelial tissue.

### 3. Compositions

Provided are compositions formulated for topical delivery of an antidepressant. In certain embodiments, the composition comprises the antidepressant as the sole pharmacologically active agent. In some embodiments, the compositions comprise an antidepressant and a beta-adrenergic receptor antagonist as first and second pharmacologically active agents, without comprising a third pharmacologically active agent. In some embodiments, the compositions comprise an antidepressant and mesenchymal stem cells (MSCs) as first and second pharmacologically active agents, without comprising a third pharmacologically active agent. In some embodiments, the compositions comprise an antidepressant, a beta-adrenergic receptor antagonist and mesenchymal stem cells as first, second and third pharmacologically active agents, without comprising a fourth pharmacologically active agent.

a. Antidepressants

Antidepressants of particular interest for use in the present compositions and methods include those antidepressants that increase extracellular levels of serotonin (5-HT). Illustrative antidepressant agents of use include without limitation selective serotonin reuptake inhibitors (SSRIs), serotonin-norepinephrine reuptake inhibitors (SNRIs), tricyclic or tetracyclic antidepressants (TCAs), a monoamine oxidase inhibitors (MAOIs) and atypical antidepressants, all of which are well known in the art.

Illustrative selective serotonin reuptake inhibitors (SSRIs) include without limitation citalopram, escitalopram, fluoxetine, norfluoxetine, fluvoxamine, fluvoxamine CR, paroxetine, paroxetine CR, and sertraline.

Illustrative serotonin-norepinephrine reuptake inhibitors (SNRIs) include without limitation desvenlafaxine, duloxetine, venlafaxine, venlafaxine XR, milnacipran, and levomilnacipran.

Illustrative tricyclic or tetracyclic antidepressants (TCAs) include without limitation amitriptyline, amoxapine, desipramine, doxepin, imipramine, nortriptyline, protriptyline, trimipramine and maprotiline.

Illustrative monoamine oxidase inhibitors (MAOIs) include without limitation selegiline, moclobemide, tranylcypromine, isocarboxazid and phenylzine.

b. Beta Adrenergic Receptor Antagonists

A wide variety of beta-adrenergic receptor antagonists are known and have been described in the scientific and patent literature, many of which are in clinical use for other conditions. Although a few exemplary antagonists are listed below, no attempt is made to identify all possible agonists and antagonists herein. Other suitable antagonists which of use can be readily identified by one of skill in the art.

In varying embodiments, the beta adrenergic receptor antagonist is selective for the β2 adrenergic receptors, affecting or antagonizing substantially only the β2 adrenergic receptors. In some embodiments, the beta adrenergic receptor antagonist is nonselective, affecting or antagonizing the β1 and β2 adrenergic receptors, the β1, β2 and β3 adrenergic receptors, or the like. It will be evident that selectivity is optionally a function of the concentration of the antagonist. For example, an antagonist can have a Ki for the β2 adrenergic receptor that is 100-fold less than its Ki for the β1 adrenergic receptor, in which example the antagonist is considered to be selective for the β2 adrenergic receptor over the β1 adrenergic receptor when used at a concentration relatively near its Ki for the β2 adrenergic receptor (e.g., a concentration that is within about 10-fold of its Ki for the β2 adrenergic receptor).

In varying embodiments, the antagonist of a beta-adrenergic receptor is a non-selective antagonist for β1 and β2 adrenergic receptors. Illustrative non-selective antagonists of beta-adrenergic receptors include without limitation, e.g., carteolol, carvedilol, dilevelol, labetalol, nadolol, penbutolol, pindolol, propranolol, sotalol, timolol, and mixtures, analogs and salts thereof. In varying embodiments, the antagonist of a beta-adrenergic receptor is a selective antagonist for β1 adrenergic receptors. Illustrative selective antagonists for β1 adrenergic receptors include without limitation from the group consisting of acebutolol, atenolol, betaxolol, bisoprolol, celiprolol, esmolol, metoprolol, nebivolol, and mixtures, analogs and salts thereof. In varying embodiments, the antagonist of a beta-adrenergic receptor is a selective antagonist for β2 adrenergic receptors. Illustrative selective antagonists for β2 adrenergic receptors include without limitation ICI 118,551 and butoxamine.

In varying embodiments, the beta adrenergic receptor antagonist can be selective or nonselective for the β2 adrenergic receptors. Similarly, in certain embodiments, the antagonist has a greater affinity for the β2 adrenergic receptors than for the β3 adrenergic receptors. Thus, in one aspect, the antagonist has a Kd for a β3 adrenergic receptor that is about 100 or more times greater than a Kd of the antagonist for a β2 adrenergic receptor. In one aspect, the antagonist is substantially free of activity as a β3 adrenergic receptor agonist, e.g., has no detectable or significant activity as a β3 adrenergic receptor agonist. For example, in some embodiments, the scaffolds and methods optionally exclude CGP 12177.

The choice of antagonist for a particular application can be influenced, for example, by factors such as the half-life of the compound, its selectivity, potential side effects, preferred mode of administration, potency, and clinical information about a given patient (e.g., any known pre-existing conditions that might be exacerbated by administration of an agonist or antagonist, potential drug interactions, or the like). Nadolol has a long half-life (on the order of 24 hours), and potentially has lower central nervous system side effects due to low lipid solubility.

c. Mesenchymal Stem Cells

In varying embodiments methods and extracellular matrices described herein entail the administration to a wound of MSCs. In some embodiments, the MSCs have been contacted and/or pre-conditioned with and/or exposed to an antidepressant and/or beta adrenergic receptor antagonist. Dasu, et al., Stem Cells Transl Med. (2014) 3(6):745-59 (hereby incorporated herein by reference in its entirety for all purposes) demonstrate that exposing MSCs to a beta adrenergic receptor antagonist promotes and/or facilitates wound healing. See also, WO 2015/031110.

The bone marrow of an adult mammal is a repository of mesenchymal stem cells (MSCs). These cells are self-renewing, clonal precursors of non-hematopoietic tissues. MSCs for use in the present methods can be isolated from a variety of tissues, including bone marrow, muscle, fat (i.e., adipose), dermis, placenta, umbilical cord, Wharton's jelly, liver and dermis, using techniques known in the art. Illustrative techniques are described in WO 2015/031110 and reported in, e.g., Chung, et al., Res Vet Sci. (2010) Nov. 12, PMID:21075407; Toupadakis, et al., American Journal of Veterinary Research (2010) 71(10):1237-1245.

Generally, the MSCs useful for administration express on their cell surface CD44 and CD90 and do not express on their cell surface CD34, CD45, CD80, CD86 or MHC-II. In various embodiments, the MSCs are adipose-derived mesenchymal stem cells (Ad-MSC). Ad-MSCs can be characterized by the surface expression of CD44, CD5, and CD90 (Thy-1); and by the non-expression of CD34, CD45, MHC class II, CD3, CD80, CD86, CD 18 and CD49d. In other embodiments, the MSCs are derived from a non-adipose tissue, for example, bone marrow, liver, dermis, placenta, umbilical cord, Wharton's jelly, lacrimal gland, and/or dermis. In some embodiments, the MSCs are non-haematopoietic stem cells derived from bone marrow (i.e., do not express CD34 or CD45).

As appropriate, the MSCs can be autologous (i.e., from the same subject), syngeneic (i.e., from a subject having an identical or closely similar genetic makeup); allogeneic (i.e., from a subject of the same species) or xenogeneic to the subject (i.e., from a subject of a different species).

In various embodiments, the MSCs may be altered to enhance the viability of the embedded, engrafted or transplanted cells. For example, the MSCs can be engineered to overexpress or to constitutively express Akt. See, e.g., U.S. Patent Publication No. 2011/0091430.

In varying embodiments, the MSCs are exposed to a concentration of antidepressant and/or beta adrenergic receptor antagonist sufficient to supersede or overcome signaling through Toll-Like receptors (e.g., TLR2) facilitating the concomitant prevention, reduction and/or inhibition of the production of IL-6 and other inflammatory mediators that inhibit wound healing. In varying embodiments, the MSCs are exposed to, cultured in or preconditioned with a concentration of at least about 0.1 μM to about 50 μM antidepressant and/or beta adrenergic receptor antagonist, e.g., from at least about 1.0 μM to about 25 μM antidepressant and/or beta adrenergic receptor antagonist, e.g., from at least about 1.0 μM to about 10 μM antidepressant and/or beta adrenergic receptor antagonist. In varying embodiments, the MSCs are exposed to, cultured in or preconditioned with a concentration of at least about 0.2 μM to about 50 μM, e.g., about 0.4 μM to about 40 μM, e.g., about 0.3 μM to about 30 μM, e.g., about 0.2 μM to about 20 μM, e.g., about 1.0 μM to about 10 μM. In varying embodiments, MSCs within the extracellular matrix may have continued exposure to the antidepressant and/or beta adrenergic receptor antagonist. In varying embodiments, the antidepressant and/or beta adrenergic receptor antagonist is added to the matrix or culture medium one or multiple times, as needed to promote, facilitate and/or accelerate wound healing.

The concentration of antidepressant and/or beta adrenergic receptor antagonist cultured with the MSCs or amount of the one or more active agents to be administered to the wound can depend on several factors, including without limitation, the nature, severity, and extent of the wound to be treated, the potency of the compound, the patient's weight, the patient's clinical history and response to the one or more active agents, and the discretion of the attending physician. Appropriate dosage can readily be determined by one of skill in the art. In varying embodiments, MSCs pre-conditioned with or exposed to antidepressant and/or beta-adrenergic receptor antagonist and embedded in an extracellular matrix or SDR are first implanted, embedded or sutured into or onto a wound, and then subsequent additional administrations of antidepressant and/or beta-adrenergic receptor antagonist are administered to the subject, e.g., either systemically administered or locally applied directly to the wound and the extracellular matrix within or on the wound.

### 4. Formulation and Delivery

The one or more active agents can be formulated for administration systemically, locally, and/or topically. For example, the one or more active agents can be formulated for administration systemically, e.g., orally or intravenously. In varying embodiments, the one or more active agents are formulated for administration topically, e.g., by application to a wound of an ointment, cream, lotion, liquid, gel, suspension, spray, foam, dressing, transdermal device, a transdermal patch, an extracellular matrix scaffold, or the like, comprising the antidepressant and optionally a beta adrenergic receptor antagonist and/or mesenchymal stem cells. As yet another example, the one or more active agents can be administered locally or intralesionally, e.g., by injecting the one or more active agents directly into tissue underlying or immediately adjacent to the wound. For example, for a skin wound, the one or more active agents can be administered by injecting it subcutaneously or intradermally at or near the site of the skin wound.

In varying embodiments, the compositions are formulated for topical administration of an antidepressant, optionally in combination with a beta adrenergic receptor antagonist and/or mesenchymal stem cells. The one or more active agents can be in the same or different pharmaceutical compositions. In varying embodiments, the pharmaceutical compositions formulated for topical administration comprise an ointment, cream, lotion, foam, liquid, gel (e.g., an aqueous gel), solution or suspension, dressing, or extracellular matrix scaffold comprising the one or more active agents, typically from about 0.001% w/v to about 1.0% w/v, 2.0% w/v, 5.0% w/v, 10.0% w/v, 15.0% w/v, 20.0% w/v, 25.0% w/v, or 30.0% w/v (weight/volume, where 1 g/100 ml is equivalent to 1%) of the one or more active agents, preferably from 0.001% w/v to 2.0% w/v, e.g., mixed with customary excipients or dissolved in an appropriate vehicle for topical application.

A variety of solid, semisolid and liquid vehicles are known in the art for topical application of agents to the skin and can find use for topical delivery of an antidepressant (e.g., an SSRI or SNRI), alone or in combination with a beta adrenergic receptor antagonist. Exemplary compositions formulated for topical application to skin can comprise an ointment (e.g., an occlusive or petrolatum-based ointment), balm, cream, lotion, gel, spray, foam, or the like, e.g., in which the one or more active agents are suspended, dissolved, or dispersed. Many suitable bases for such ointments, creams, lotions, gels, etc. are known in the art and can be used. At least one component of the composition is optionally insoluble in water and/or hydrophobic; for example, the composition optionally includes an oil (e.g., a suspension of an oil in water), petrolatum, a lipid, or the like. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, N.Y.: Springer; 107-174 (1971), and Hadgcraft, “Recent progress in the formulation of vehicles for topical applications,” Br J Dermatol., 81:386-389 (1972). It is presumed that the person of skill is familiar with these various vehicles and preparations and they need not be described in detail herein.

An antidepressant (e.g., an SSRI or SNRI), alone or in combination with a beta adrenergic receptor antagonist can be mixed into such modalities (ointments, creams, lotions, gels, foams, etc.) for topical administration. In general, the concentration of the agents provides a gradient which drives the one or more agents into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, Fla., 1997).

In some embodiments, the antidepressant (e.g., an SSRI or SNRI), alone or in combination with a beta adrenergic receptor antagonist, is formulated in a cream. Typically, the cream comprises one or more hydrophobic lipids, with other agents to improve the “feel” of the cream or to provide other useful characteristics. In one embodiment, for example, a cream may contain 0.01 mg to 10 mg of antidepressant, with or without one or more a beta adrenergic receptor antagonist, per gram of cream in a white to off-white, opaque cream base of purified water USP, white petrolatum USP, stearyl alcohol NF, polyethylene glycol, propylene glycol USP, polysorbate 60 NF, cetyl alcohol NF, and benzoic acid USP 0.2% as a preservative. In one embodiment, the antidepressant (e.g., an SSRI or SNRI), alone or in combination with a beta adrenergic receptor antagonist, can be mixed into a commercially available cream, e.g., Vanicream™ (Pharmaceutical Specialties, Inc., Rochester, Minn.) comprising purified water, white petrolatum, cetearyl alcohol and ceteareth-20, sorbitol solution, propylene glycol, simethicone, glyceryl monostearate, polyethylene glycol monostearate, sorbic acid and BHT.

In other embodiments, the antidepressant (e.g., an SSRI or SNRI), alone or in combination with a beta adrenergic receptor antagonist, is formulated in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri (PPG-3 myristyl ether) citrate, disodium EDTA, methylparaben, ethylparaben, propylparaben, xanthan gum, butylparaben, and methyldibromo glutaronitrile.

In some embodiments, the antidepressant (e.g., an SSRI or SNRI), alone or in combination with a beta adrenergic receptor antagonist, is formulated in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

In whatever topical formulation in which the antidepressant (e.g., an SSRI or SNRI), alone or in combination with a beta adrenergic receptor antagonist, is formulated is administered (that is, whether by lotion, gel, spray, etc.), in varying embodiments they can administered at a therapeutically effective dosage, e.g., in the range of about 0.01 mg to 10 mg, or in the range of about 0.01 μM to about 10 μM per 10 cm².

In varying embodiments, the antidepressant (e.g., an SSRI or SNRI), alone or in combination with a beta adrenergic receptor antagonist, can be introduced into the bowel by use of a suppository. As is known in the art, suppositories are solid compositions of various sizes and shapes intended for introduction into body cavities. Typically, the suppository comprises a medication, which is released into the immediate area from the suppository. Typically, suppositories are made using a fatty base, such as cocoa butter, that melts at body temperature, or a water-soluble or miscible base, such as glycerinated gelatin or polyethylene glycol.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the unit dosage forms of the topical formulations described herein will depend on (a) the unique characteristics of the active material and the particular effect to be achieved and (b) the limitations inherent in the art of compounding such an active material for use in humans and animals, as disclosed in detail in this specification, these being features of the present methods.

In varying embodiments, the topically formulated antidepressant is co-administered with a beta adrenergic receptor antagonist that is administered by a route other than topical. In a pharmaceutical composition for oral, sublingual, subcutaneous, intramuscular, intravenous, transdermal, or local administration, for example, the one or more active agents can be administered in unit forms of administration, either as such, for example in lyophilized form, or mixed with conventional pharmaceutical carriers. Appropriate unit forms of administration include oral forms such as tablets, which may be divisible, gelatin capsules, powders, granules and solutions or suspensions to be taken orally, sublingual and buccal forms of administration, subcutaneous, intramuscular or intravenous forms of administration, and local forms of administration.

When a solid composition is prepared in the form of tablets, the main active ingredient is optionally mixed with a pharmaceutical vehicle such as gelatin, starch, lactose, magnesium stearate, talcum, gum arabic or the like. The tablets can be coated with sucrose or other appropriate substances, or can be treated so as to have a prolonged or delayed activity and so as to release a predetermined amount of active principle continuously. A preparation in the form of gelatin capsules can be obtained by mixing the active ingredient with a diluent and pouring the resulting mixture into, soft or hard gelatin capsules. A preparation in the form of a syrup or elixir optionally contains the active ingredient together with a sweetener, antiseptic, flavoring and/or appropriate color. Water-dispersible powders or granules can contain the active ingredient mixed with dispersants, wetting agents or suspending agents, as well as with sweeteners or taste correctors. Suppositories (e.g., for vaginal or rectal administration) can be prepared with binders melting at the appropriate (e.g., vaginal or rectal) temperature. Parenteral administration is typically effected using aqueous suspensions, saline solutions or injectable sterile solutions containing pharmacologically compatible dispersants and/or wetting agents. The one or more active agents is optionally encapsulated in liposomes or otherwise formulated for prolonged or delayed release, e.g., whether for topical, local, and/or systemic administration.

Follow-up administrations of the one or more active agents can be administered to the patient at one time or over a series of administrations, as appropriate. For repeated administrations over several days or longer, depending on the condition, the treatment is optionally sustained until a desired result occurs; for example, until a wound is healed. Similarly, treatment can be maintained as required. The progress of the therapy can be monitored by conventional techniques and assays.

### 5. Extracellular Matrix Scaffolds

In various embodiments, embedding, engraftment or transplantation of the antidepressant and/or beta adrenergic receptor antagonist-conditioned MSCs is facilitated using a matrix or caged depot, e.g., an extracellular matrix scaffold. For example, the MSCs can be embedded, engrafted or transplanted in a “caged cell” delivery device wherein the cells are integrated into a biocompatible and/or biologically inert matrix (e.g. a hydrogel or other polymer or any device) that restricts cell movement while allowing the cells to remain viable. Synthetic extracellular matrix and other biocompatible vehicles for delivery, retention, growth, and differentiation of stem cells are known in the art and find use in the present methods. See, e.g., Prestwich, J Control Release. 2011 Apr. 14, PMID 21513749; Perale, et al., Int J Artif Organs. (2011) 34(3):295-303; Suri, et al., Tissue Eng Part A. (2010) 16(5):1703-16; Khetan, et al., J Vis Exp. (2009) Oct. 26; (32). pii: 1590; Salinas, et al., J Dent Res. (2009) 88(8):681-92; Schmidt, et al., J Biomed Mater Res A. (2008) 87(4):1113-22 and Xin, et al., Biomaterials (2007) 28:316-325. The extracellular matrix can be naturally occurring (e.g., decellularized tissue) or synthetic.

Any biocompatible, biodegradable matrices known in the art can be used as a scaffold or extracellular matrix for the MSCs. In varying embodiments, the matrix is made of naturally derived components (e.g., collagen, elastin, laminin, gelatin and/or other naturally derived materials). In varying embodiments, the matrix can be synthetic or made of or comprise non-naturally derived components. Biocompatible, biodegradable materials useful in the matrices include, e.g., polyglycolic acid (PGA), type 1 collagen, Poly-DL-lactide-caprolactone (PCL), laminin, gelatin, chitin, alginate, keratin, and the like. In varying embodiments, the matrix comprises collagen. Illustrative extracellular matrices that are commercially available and find use include without limitation, e.g., matrices available from Integra Life Sciences (integralife.com); Oasis Wound matrices available from Cook Biotech (oasiswoundmatrix.com); MatriStem matrices from ACell (acell.com); GRAFTJACKET® matrices from Wright Medical Technology (wmt.com); MatriDerm® matrices by MedSkin Solutions Dr. Suwelack AG (medskin-suwelack.com); and UNITE™ Biomatrices by Baxter Healthcare (synovissurgical.com).

As appropriate or desired, the embedded, engrafted or transplanted antidepressant and/or beta adrenergic receptor antagonist-conditioned MSCs can be modified to facilitate retention of the MSCs at the region of interest or the region of delivery, e.g., at the site of the wound. In other embodiments, the region of interest for embedding, engraftment or transplantation of the cells is modified in order to facilitate retention of the MSCs at the region of interest or the region of delivery. In one embodiment, this can be accomplished by introducing stromal cell derived factor-1 (SDF-1) into the region of interest, e.g., using a linkage chemistry or integrated biodegradable matrix (e.g., Poly(D,L-lactide-co-glycolide (PLGA) beads) that would provide a tunable temporal presence of the desired ligand up to several weeks. MSCs bind to the immobilized SDF-1, thereby facilitating the retention of MSCs that are delivered to the region of interest for embedding, engraftment or transplantation. In other embodiments, integrating cyclic arginine-glycine-aspartic acid peptide into the region of interest can facilitate increased MSC binding and retention at the region of interest for embedding, engraftment or transplantation. See, e.g., Ratliff, et al., Am J Pathol. (2010) 177(2):873-83.

In varying embodiments, the one or more active agents are directly attached to the extracellular scaffold matrix, e.g., via covalent bonding or crosslinking. Crosslinkers of use are known in the art. In varying embodiments, the beta adrenergic receptor antagonist is directly attached or crosslinked to the extracellular scaffold matrix using a linkage chemistry or integrated biodegradable matrix (e.g., Poly(D,L-lactide-co-glycolide (PLGA) beads).

In varying embodiments, at least about 0.25×10⁶ MSCs are provided to the subject, e.g., in the matrix embedded, engrafted or implanted at the site of the wound. As appropriate, the number of MSCs injected into the subject or embedded, engrafted or implanted into the matrix at the site of the wound can be at least about, e.g., 1×10⁴ cells, 2.5×10⁴ cells, 5×10⁴ cells, 7.5×10⁴ cells, 1×10⁵ cells, 2.5×10⁵ cells, 5×10⁵ cells, 7.5×10⁵ cells, 1×10⁶ cells, 2.5×10⁶ cells, 5×10⁶ cells, 7.5×10⁶ cells, 1×10⁷ cells, 2.5×10⁷ cells, 5×10⁷ cells, 7.5×10⁷ cells, or 1×10⁸ cells.

In various embodiments, the cells can be delivered or embedded in the extracellular matrix at a concentration in the range of about 1×10⁶ cells/ml to about 1×10⁸ cells/ml, for example, in the range of about 5×10⁶ cells/ml to about 5×10⁷ cells/ml, for example about 1×10⁶ cells/ml, 5×10⁶ cells/ml, 1×10⁷ cells/ml, 5×10⁷ cells/ml or 1×10⁸ cell s/ml.

The total amount of cells that are envisioned for use depend upon the desired effect, patient state, and the like, and may be determined by one skilled within the art. Dosages for any one patient depends upon many factors, including the patient's species, size, body surface area, age, the particular MSCs to be administered, sex, scheduling and route of administration, general health, and other drugs being administered concurrently.

### 6. Wound Dressings

Further provided are wound dressings. In varying embodiments, the wound dressing comprises the compositions or the extracellular matrix scaffolds, as described above and herein. In some embodiments, the wound dressing can be impregnated with the compositions or the extracellular matrix scaffolds, or at least one surface of the dressing can be coated with the compositions or the extracellular matrix scaffolds. The composition is optionally formulated for slow, controlled release of the one or more active agents. The dressing can be a bulky dressing, a pad, a bandage, a self-adhesive bandage, or other suitable biocompatible dressing. A related class of embodiments provides a transdermal device or a transdermal patch comprising the composition. The embodiments of the compositions and extracellular matrix scaffolds are as described above and herein.

### 7. Methods of Increasing Epithelial Wound Healing

In one aspect, provided are methods for increasing the rate of repair of wounds in epithelial tissues, e.g., in humans. The methods involve topical administration of an antidepressant as sole pharmacologically active agent or in combination with an beta adrenergic receptor antagonist and/or mesenchymal stem cells to stimulate wound repair (e.g., re-epithelialization of the area), e.g., by stimulating migration and/or proliferation of epithelial cells (e.g., of keratinocytes for repair of a wound in the skin). The embodiments of the active agents are as described above and herein. In varying embodiments, the one or more active agents are delivered to the wound in a wound dressing or extracellular matrix scaffold.

Certain embodiments provide methods for increasing and/or promoting the rate of wound healing and/or wound contraction and/or decreasing the area of a wound in a target patient. In the methods, the target patient is by identifying a person comprising or at risk for comprising a wound in an epithelial tissue, and an effective amount of an antidepressant, alone or in combination with a beta adrenergic receptor antagonist and/or mesenchymal stem cells is topically administered to the target patient.

In varying embodiments, the wound is in skin, e.g., is a cutaneous wound. The methods can be particularly useful for stimulating healing of chronic, non-healing skin wounds. Thus, in some embodiments, the wound comprises a chronic skin wound, e.g., a venous stasis ulcer, a diabetic foot ulcer, a neuropathic ulcer, a digital ulcer or a decubitus ulcer. Other exemplary chronic wounds for which the methods can be used include, but are not limited to, other chronic ulcers such as immune-mediated (e.g., rheumatoid arthritis) ulcers, radiotherapy-induced ulcers, and ulcers resulting from digital ischemia (e.g., Reynaud's phenomenon), vasospasm, vasculitis, arteriolar obstruction or occlusion, pyoderma gangrenosum, thalessemia, connective tissue disorders (e.g., scleroderma) and other dermatologic diseases that result in non-healing wounds. In some embodiments, the wound results from surgical wound dehiscence.

The methods can also be applied to other types of wounds. For example, the wound can comprise a burn, cut, incision, laceration, ulceration, abrasion, or essentially any other wound in an epithelial tissue. Similarly, the methods can be applied to repair of wounds in essentially any epithelial tissue, including, but not limited to, skin, a genitourinary epithelium, a gastrointestinal epithelium, a pulmonary epithelium, or a corneal epithelium. In varying embodiments, the epithelial tissue is other than an epithelial tissue comprising an eye.

In varying embodiments, the active agent or agents can be topically administered by application to the wound an ointment, cream, lotion, liquid, gel, suspension, spray, foam, or the like comprising the one or more active agents. In some embodiments, the one or more active agents can be topically administered by application of a dressing comprising the antagonist to the wound, e.g., a dressing impregnated with the one or more active agents or having at least one surface coated with the one or more active agents, e.g., a pad or self-adhesive bandage.

In some embodiments, the one or more active agents can be topically administered by application of a transdermal device or a transdermal patch. Either “passive” or “active” transdermal devices can be employed for administration of one or more compositions described herein, the selection of which will depend in part upon the location for application of the device (e.g., at or proximal to the site of epithelial damage for local administration of, for example, rapidly metabolized compositions, or distal to the site for systemic composition administration). Examples of passive transdermal devices include reservoir-type patches (e.g., in which the composition is provided within a walled reservoir having a permeable surface) and matrix-type patches (in which the composition is dispersed within a polymeric composition). Active transdermal devices include, but are not limited to, devices employing iontophoresis (e.g., a low voltage electrical current), electroporation (e.g., short electrical pulses of higher voltage), sonophoresis (e.g., low frequency ultrasonic energy), or thermal energy for delivery of the composition. Typically, passive-type transdermal devices would be utilized for application at a current site of epithelial damage, since additional mechanisms for overcoming the epithelial barrier provided by active-type transdermal devices is not necessary. For a review of various transdermal technologies, see Ghosh, Pfister and Yum Eds. (1997) Transdermal and Topical Drug Delivery Systems (CRC Press, London); Potts and Guy (Eds.) (1997) Transdermal Drug Delivery (Marcel Dekker, New York); and Potts and Cleary (1995) Transdermal drug delivery: useful paradigms. J Drug Targ. 3:247-251.

In varying embodiments, the one or more active agents are topically administered by introduction of a foam (e.g., a biologically inert or pharmaceutically acceptable foam). In varying embodiments, the foam comprising the one or more active agents is administered to an epithelial-lined cavity comprising the wound, e.g., an oral, vaginal, or bladder cavity.

In varying embodiments, two or more active agents can be administered via multiple routes of administration. Thus, for example, two or more active agents can be administered both topically and orally or topically and by injection, simultaneously or sequentially, as indicated by the nature and severity of the wound to be treated.

In varying embodiments, administration of the one or more active agents is prophylactic; e.g., the one or more active agents can be administered to a patient at risk for developing a wound or who has a visible incipient wound (e.g., a patient having diabetic neuropathy). Thus, in some embodiments, the one or more active are administered prior to full development of a wound or at the time of wounding. More typically, however, the one or more active agents are administered after the wound is created, e.g., after the patient presents to a physician for treatment of a wound or chronic wound.

In varying embodiments, the methods can increase the rate of wound healing by a statistically significant amount. In some embodiments, the rate of wound healing in the target patient treated with an antidepressant, alone or in combination with a beta adrenergic receptor antagonist and/or mesenchymal stem cells is at least about 10% greater than in a corresponding untreated individual (e.g., at least about 15% greater or at least about 20% greater).

Further, topical administration of an antidepressant, alone or in combination with a beta adrenergic receptor antagonist and/or mesenchymal stem cells can improve healing of burns. In some embodiments, the burn patient does not display hypermetabolic syndrome or a hypermetabolic response. Hypermetabolic syndrome, described in the literature, can occur with burns covering greater than 40% of the patient's total body surface area. In one aspect, the burn covers less than about 80% of the patient's total body surface area, e.g., less than about 70%, 60%, or 50% of the patient's total body surface area. In one class of embodiments, the burn covers less than about 40% of the patient's total body surface area, optionally less than about 35%, less than about 30%, less than about 20%, or even less than about 10% or less than about 5% of the patient's total body surface area. It will be evident that the area covered by the burn can be continuous or discontinuous. The patient may or may not display a hypermetabolic response.

### 8. Methods of Monitoring

Clinical efficacy can be monitored using any method known in the art. Measurable parameters to monitor efficacy will depend on the condition being treated. For monitoring the healing or contraction of an epithelial wound, both subjective parameters (e.g., patient reporting; character of the wound) and objective parameters (e.g., area of the wound, depth of the wound, tissue involved, wound edges, perilesional maceration, tunneling, type of tissue in the wound bed, exudate, infection, inflammation, biofilm formation, peripheral blood flow and pain) can be evaluated. Symptoms for patients epithelial wounds can be measured and quantified using appropriate assays established in the art, e.g., as described in Restrepo-Medrano, et al., EWMA Journal (2012) 12(2):39-45. Behavioral changes in the subject (e.g., attitude, mood, appetite, grooming, sociability, energy levels, increased activity levels, weight gain/loss, mobility, exhibition of increased comfort) may also be relevant to the evaluation of epithelial wound healing. These parameters can be measured using any methods known in the art. In varying embodiments, the different parameters can be assigned a score. Further, the scores of two or more parameters can be combined to provide an index for the subject.

Observation of the stabilization, improvement and/or reversal of one or more symptoms or parameters by a measurable amount (e.g., increased wound contraction, decreased wound area, decreased wound depth, decreased exudate, decreased inflammation, lack of infection, decreased pain; eliminated wound infection) indicates that the treatment or prevention regime is efficacious. Observation of the progression, increase or exacerbation of one or more symptoms indicates that the treatment or prevention regime is not efficacious. For example, in the case of evaluating wound healing after one or more topical administrations of an antidepressant alone or in combination with a beta adrenergic receptor antagonist and/or mesenchymal stem cells indicates that the treatment or prevention regime is efficacious. Likewise, observation of reduction or decline, lack of improvement or worsening of one or more symptoms or parameters (e.g., same or increased wound area; same or increased wound depth, same or increased exudate, same or increased inflammation, same or increased pain, persistent wound infection) indicates that the treatment or prevention regime is not efficacious.

In certain embodiments, the monitoring methods can entail determining a baseline value of a measurable biomarker (e.g., inflammatory markers including TNFα, IL-6, IFNγ, reactive oxygen species, IL-1, FasL, and IL-8 or anti-inflammatory markers including IL-10 or TGFβ) or clinical parameter (e.g., area of the wound, depth of the wound, tissue involved, wound edges, perilesional maceration, tunneling, type of tissue in the wound bed, exudate, infection, inflammation, biofilm formation, peripheral blood flow and pain) in a subject before administering a dosage of the one or more active agents described herein, and comparing this with a value for the same measurable biomarker or parameter after a course of treatment.

In other methods, a control value (i.e., a mean and standard deviation) of the measurable biomarker or parameter is determined for a control population. In certain embodiments, the individuals in the control population have not received prior treatment and do not have the disease condition subject to treatment, nor are at risk of developing the disease condition subject to treatment (e.g., do not have and are not at risk of developing a chronic or non-healing epithelial wound; e.g., do not have diabetes, a vascular disorder or a connective tissue disorder). In such cases, if the value of the measurable biomarker or clinical parameter approaches the control value, then treatment is considered efficacious. In other embodiments, the individuals in the control population have not received prior treatment and have been diagnosed with the disease condition subject to treatment (e.g., have a chronic or non-healing epithelial wound; have diabetes, a vascular disorder or a connective tissue disorder). In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

### 9. Kits

Further provided are kits. In varying embodiments, the kits include one or more pharmaceutical compositions comprising a topically formulated antidepressant, optionally in combination with a topically formulated beta adrenergic receptor antagonist, as described above and herein. The one or more active agents can be in the same or different compositions, and can be packaged in one or more containers, e.g., bottle, vial, spray or aerosol can, or other suitable container. In varying embodiments, the pharmaceutical compositions are provided in unitary dosages. The kits can further provide instructions for administering the composition or compositions to a patient suffering a wound in an epithelial tissue. In varying embodiments, the kits can comprise one or more extracellular matrix scaffolds or wound dressings, as described above and herein.

### EXAMPLES

The following examples are offered to illustrate, but not to limit the claimed invention.

### Example 1

Treatment of Chronic, Non-Healing Wounds by Locally Increasing Serotonin Levels

### Methods:

Protocols Approved.

Institutional Review Board (IRB) approval was obtained for collecting human neonatal foreskin discarded at the time of circumcision. Institutional Animal Care and Use Committee (IACUC) approval was obtained for all animal experiments, which were carried out according to approved protocols.

Primary Neonatal Human Keratinocyte (NHK) Isolation and Culture.

NHKs were isolated and cultured from human foreskin as previously reported [1, 2]. NHKs from at least 3 different donors passage 4-6 were reported for each experiment.

Scratch Wound Assays.

NHKs from 3 different donors were plated on collagen-coated 12-well plates at fully confluent density and treated with Mitomycin C to prevent proliferation. P-200 tips were used to induce a scratch wound in the middle of each well and time-lapse images were recorded every 30 minutes for 6 hours and % healed were calculated as following: % Healed=([(SA)]_(t=0)−[(SA)]_(t=6))/[(SA)]_(t=0) which SA represents surface area of the scratch wound gap at 0-hour (t=0) or 6-hour time point. Data from 3 different lines of NHKs were normalized and the average of each treatment group was reported.

Keratinocyte Proliferation.

MTS assays (CellTiter 96, Catalog G3582, Promega, Sunnyvale, Calif.) were performed according manufacturer's protocol to assess proliferation rate of NHKs from 3 different donors under different concentrations of 5-HT. Briefly, 5×10³ cells were seeded per well in 96-well plates. Standard curves were generated with different cell densities ranging from 5×10² to 2×10⁴ cells/well. Sample wells were treated in triplicate with either PBS vehicle or different concentrations of 5-HT ranging from 10 nM, 100 nM to 1 μM. Cell growth was analyzed every 24 hours for a total of 72 hours. Data from different time points were normalized to 0-hour.

Multiplex Assays.

NHKs from 3 different donors were cultured in 100 mm dishes until 80% confluent and treated with 10 μM 5-HT for 6 hours. Protein lysates were collected and quantified with Bradford assays before proceeding according to manufacturer's protocol (Millipore Multiplex Assay (MILLIPLEX® MAP 48-680MAG). Results were normalized to total protein levels quantified separately by Bradford protein assays.

Fluoxetine ELISA.

Whole blood from mice treated with topical fluoxetine or vehicle control were collected via cardiac puncture. Whole blood was centrifuged at 1,500×g for 15 minutes and collected serum was stored in plastic tube at 80° C. until assayed. The concentration of fluoxetine present in serum was quantified using ELISA (Neogen Corporation, Catalog #107619) adapted from manufacturer's protocol [3]. Briefly, serum from untreated mice was spiked with the addition of fluoxetine hydrochloride at 0.49, 1.95, 7.8, 31.25, 125, and 500 ng/ml to create the standard curve. Serum samples were diluted at 1:5. Drug-enzyme complex was detected using K-Blue substrate (TMB). Red Stop solution was used to halt the reaction and the extent of color development was quantified with Biotek plate reader at 650 nm.

Mixed Lymphocyte Reactions

Mixed lymphocyte reactions were performed as described [4]. Briefly, allogenic effector B6 CF SE-labeled T cells were incubated in a 96-well plate (250,000/well) with CD11c+ Bal/b APC (100,000/well) that were purified from bulk splenocytes on anti-CD11c-coupled Milteny microbeads. 1 μM fluoxetine of 10 μM serotonin was additionally added to co-cultures. Proliferation was measured based on CFSE dilution.

In Vivo Wounding Model.

Diabetic status (blood glucose >300 mg/dl) was confirmed in female Db/Db mice at age 11 weeks. At age 13 weeks, mice underwent hair removal and synchronization of follicle cycling through depilation and shaving. Synchronization was confirmed based on lack of skin pigmentation in included mice. 1 day after synchronization, 13-week-old Db/Db female mice underwent full-thickness excisional wounding using a splinted model as described [5]. Briefly, 8 mm punch biopsies were used to make full-thickness excisional wounds. 10 mm silicone splints were sutured around the perimeter of wounds to prevent contraction.

In Vivo Treatments.

Mice were wounded as described above. To study the role of topical fluoxetine and topical serotonin in wound healing, mice (5 per group) were treated daily with 30 μl of a topical solution of either 0.2% fluoxetine or 2% serotonin dissolved in a 5% polyethylene glycol vehicle or control vehicle alone. At day 10, mice were sacrificed and wounds were harvested to assess re-epithelialization.

To study the role of combinatorial topical fluoxetine and topical timolol therapy in wound healing, mice (5 per group) were treated daily 30 μl of a topical solution of either 0.2% fluoxetine alone, 0.2% fluoxetine plus 0.01% timolol, 0.01% timolol, or control vehicle alone. At day 12, mice were sacrificed and wounds were harvested to assess re-epithelialization.

To study the role of combinatorial topical fluoxetine and MSC in wound healing, all mice received INTEGRA™ matrix scaffolds. Mice were then divided into four different treatment groups (5 mice per group). Two group received un-seeded INTEGRA™ alone, one of which received daily doses of 1 μM fluoxetine, the other received PBS daily. One group received INTEGRA™ pre-seeded with 250,000 MSC and was treated daily with PBS. One group received INTEGRA™ pre-seeded with 250,000 MSC pre-treated with fluoxetine and daily doses of 1 μM fluoxetine.

Re-Epithelialization Analysis.

Following euthanasia of mice, wounds were excised with a 2 mm rim of unwounded tissue and bisected. Wound tissue was fixed in paraformaldehyde for 12-16 hours and then paraffin embedded. Serial 5 μM sections were obtained from the center of the wound and the section with the largest wound bed was stained with hemotoxylin and eosin. Images were taken using 10× objective on a Keyance microscope. Re-epithelialization was determined on sections created. Image J analysis software was utilized to divide the re-epithelialized tissue by the entire original wound surface. N=5 mice/group.

Immunohistochemistry:

Following euthanasia, wound tissue was harvested and processed as described above. Antibodies: F4/80 (Serotec, now BioRad MCA497A488, clone CI:A3-1, 1:100 dilution); GR-1 (Biolegend, catalog number 108402, clone RB6-8C5, 1:200 dilution); CD31 (Santa Cruz, catalog number sc1506, clone M20, 1:1800 dilution). Wound beds were analyzed in Spectrum software (UC Davis). Wound beds were outlined based on subcutaneous fat presence and positive cells were calculated as #/mm² of wound bed.

In Vitro Polarization.

Wild type C57BL/6 male and female mice were used. Bone marrow-derived (BMD) cells were isolated from mouse femurs and tibias. ACK solution (Thermo Fisher catalog 50-983-220) was used for 5 minutes at 37° C. to lyse red blood cells. BMD macrophages (BMDMs) were matured with (20 ng/mL) M-CSF (R&D) for 7 days. On day 7, BMDM phenotype was confirmed with flow cytometry by CD11b and F4/80 markers (Biolegend). Following maturation, BMDMs were either polarized towards M1 lineage with 10 ng/ml LPS (Sigma Aldrich) and 100 ng/ml IFN-g (R&D) or M2 lineage with 20 ng/ml IL-4 (PeproTech) and IL-13 (R&D) in cRPMI (1×RPMI (Cellgro), 10% FBS, 1× sodium pyruvate (GIBCO), 1× penicillin/streptomycin (GIBCO) at 37° C. for 24 hours. Non-polarized BMDMs and M1-polarized BMDMs were used as experimental controls for polarization procedure. M2-polarized BMDMs were co-treated with either PBS or FLX and/or 100 nM NAN-190 or 1 μM Ketanserin. Cells were either collected after 4 hours by scraping and stored in buffer RLT (Qiagen) at −80° C. or were read with flow cytometry after 24 hours and data were appropriately compensated and analyzed by FlowJo.

RNA Extraction from Mouse Tissue:

Tissue was lysed using mortar and pestle in the presence of liquid nitrogen and then homogenized using Qiazol (Qiagen) followed by RNeasy Miniprep (Qiagen). RNA was reverse-transcribed to cDNA using iScript Reverse Transcription Supermix (Bio-Rad).

RT-PCR:

RT-PCR was performed using PowerUp SYBR Green Master Mix (Thermo/Applied Biosystems). Data was analyzed using the ΔΔCt method and normalized using geometric mean of the housekeeping genes (GAPDH, Hsp90, 18S rRNA). Fold change is shown relative to control groups.

Primer Sequences:

| Target | Forward | Reverse |
|---|---|---|
| IL-10 | GCTCTTACTGACTGGCATGAG | CGCAGCTCTAGGAGCATGTG |
| IL-6 | CCAAGAGGTGAGTGCTTCCC | CTGTTGTTCAGACTCTCTCCCT |
| NOS2 | CAGCTGGGCTGTACAAACCTT | CATTGGAAGTGAAGCGTTTCG |
| Arg-1 | AGGAGCTGTCATTAGGGACATC | CTCCAAGCCAAAGTCCTTAGAG |
| CD206 | CTCTGTTCAGCTATTGGACGC | CGGAATTTCTGGGATTCAGCTTC |
| α-SMA | TCAGGGAGTAATGGTTGGAATG | GGCAGTAGTCACGAAGGAATAG |
| GAPDH | GGAGAAACCTGCCAAGTATGA | CCTGTTGCTGTAGCCGTATT |
| 18S rRNA | CCCAACTTCTTAGAGGGACAAG | GCTTATGACCCGCACTTACT |
| Hsp90 | AAACAAGGAGATTTTCCTCCGC | GTCCAGGGCATCTGAAGCATT |
| Ym-1 | CAGGTCTGGCAATTCTTCTGAA | GTCTTGCTCATGTGTGTAAGTGA |

| | |
|---|---|
| TGF-β, | Validated proprietary primers from Qiagen |
| IL-1β, and | |
| TNF-α | |

Statistical Analysis.

Student-t tests were performed to identify statistical significance between each individual treatment group against the control group. P values <0.05 are considered significant.

### Results/Discussion:

Bone Marrow-Derived MSC (BMSC) Produce Serotonin (5HT) and Serotonin Increases BMSC Proliferation and Migration.

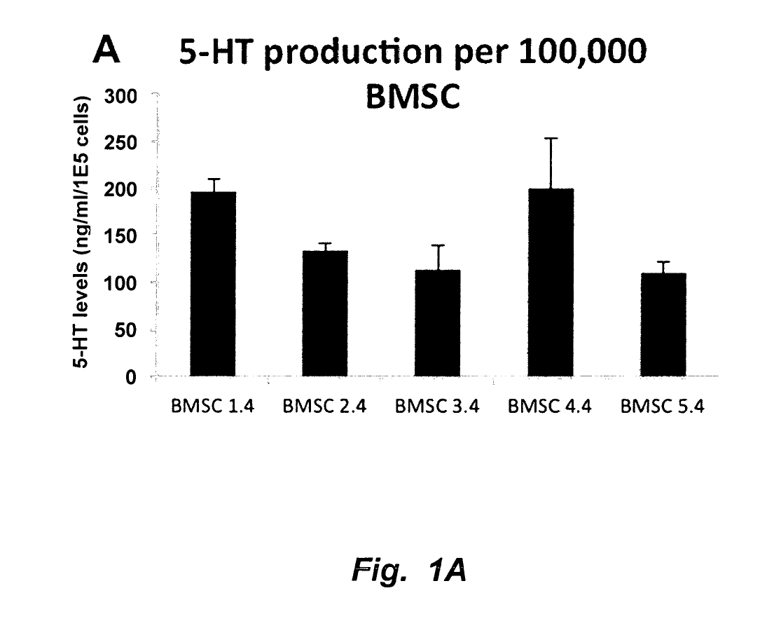

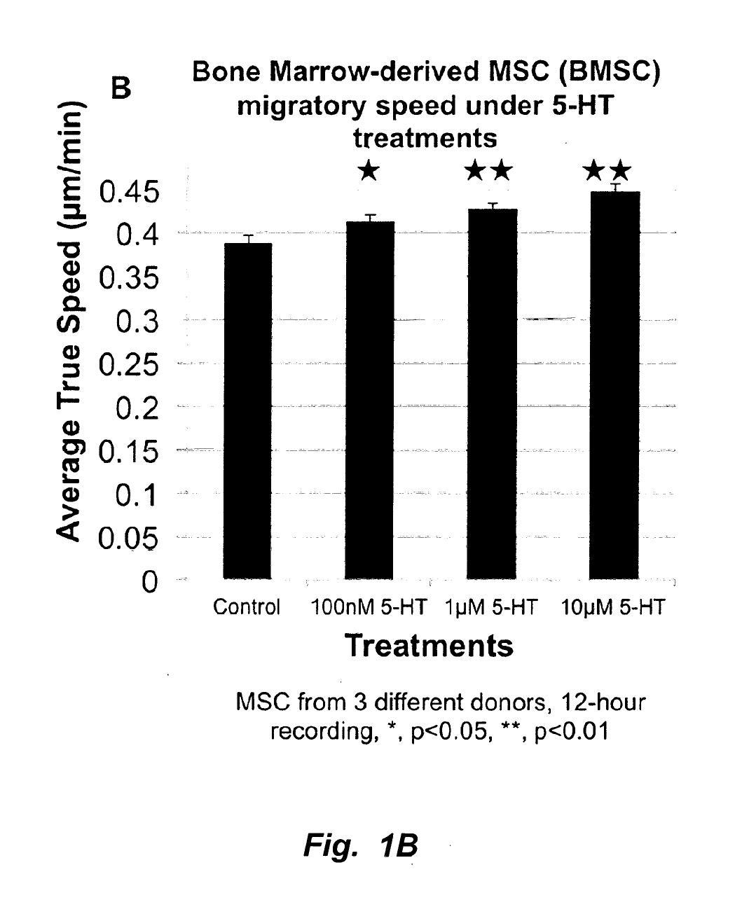

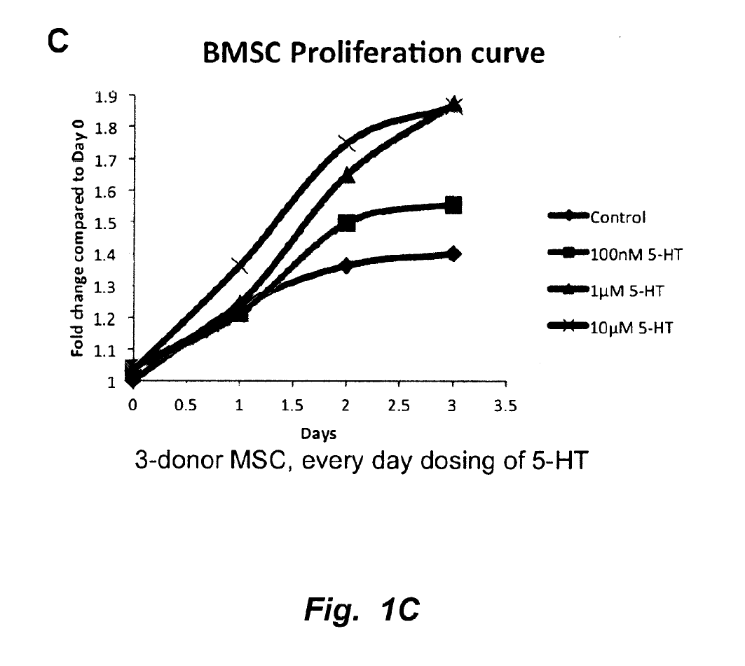

BMSC derived from 5 different donors produce serotonin at a level ranging from 100-200 ng/mL per 100,000 cells over a 48-hour incubation period (FIG. 1A). BMSC were then pooled from 3 different donors. 10 uM 5HT significantly increased BMSC migratory speed and proliferation as compared to untreated BMSC (FIGS. 1B and 1C). This finding provides evidence that the serotonergic pathway may play a role in recruiting MSC to the wound site.

Serotonin and Fluoxetine Increase Keratinocyte Migration In Vitro.

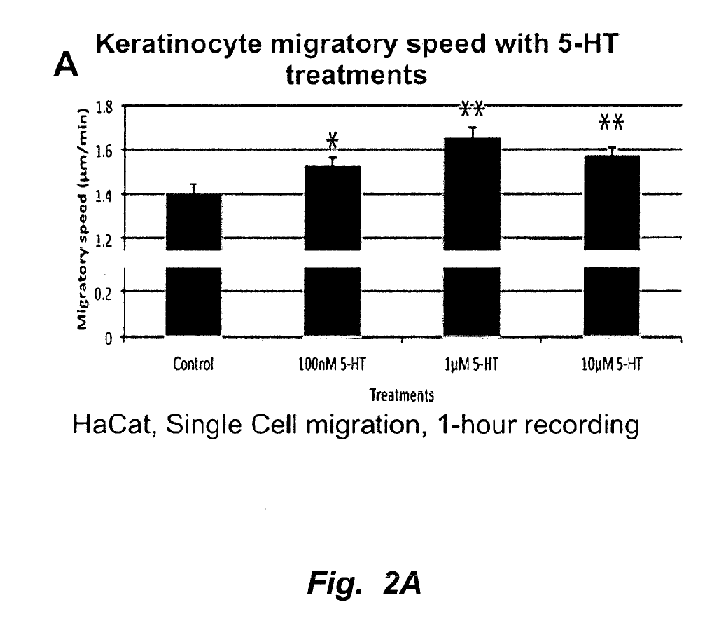

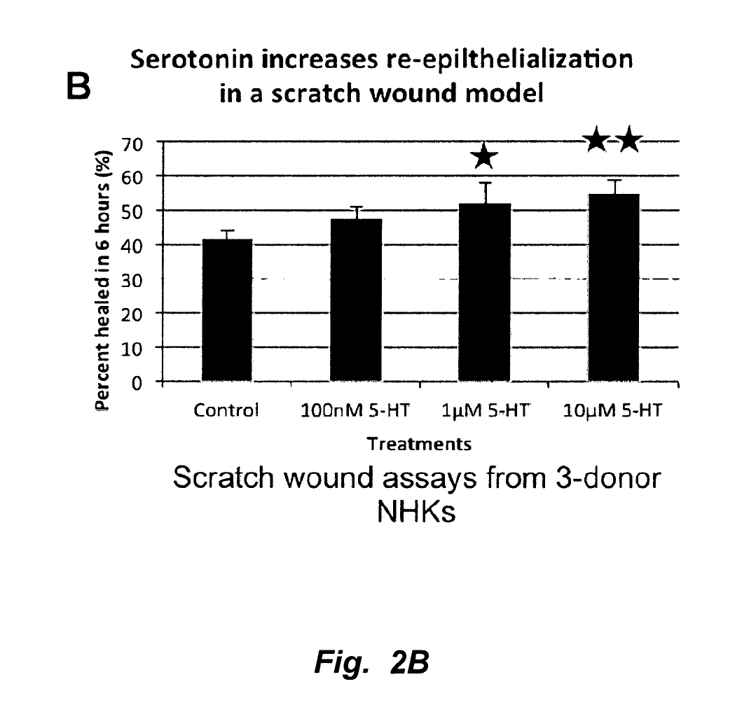

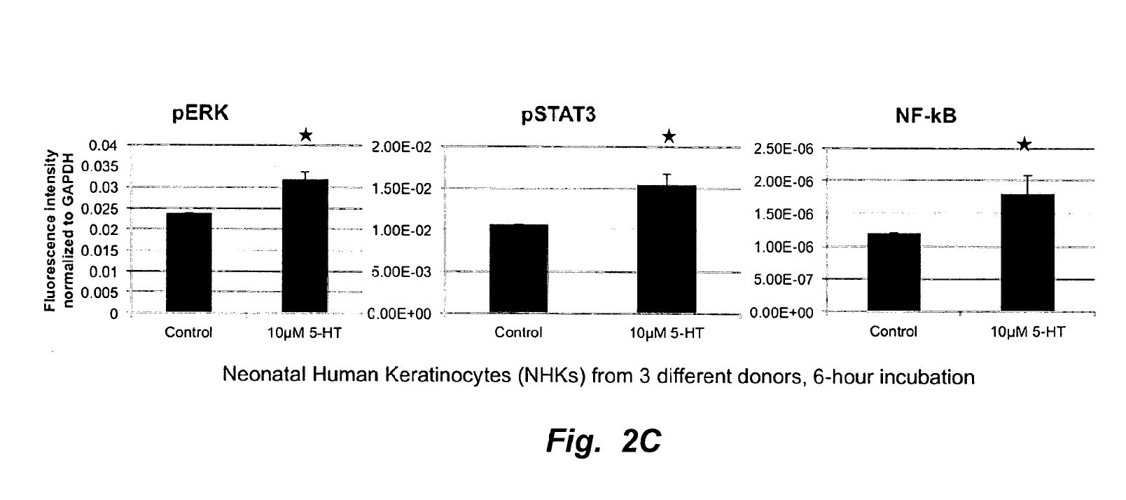

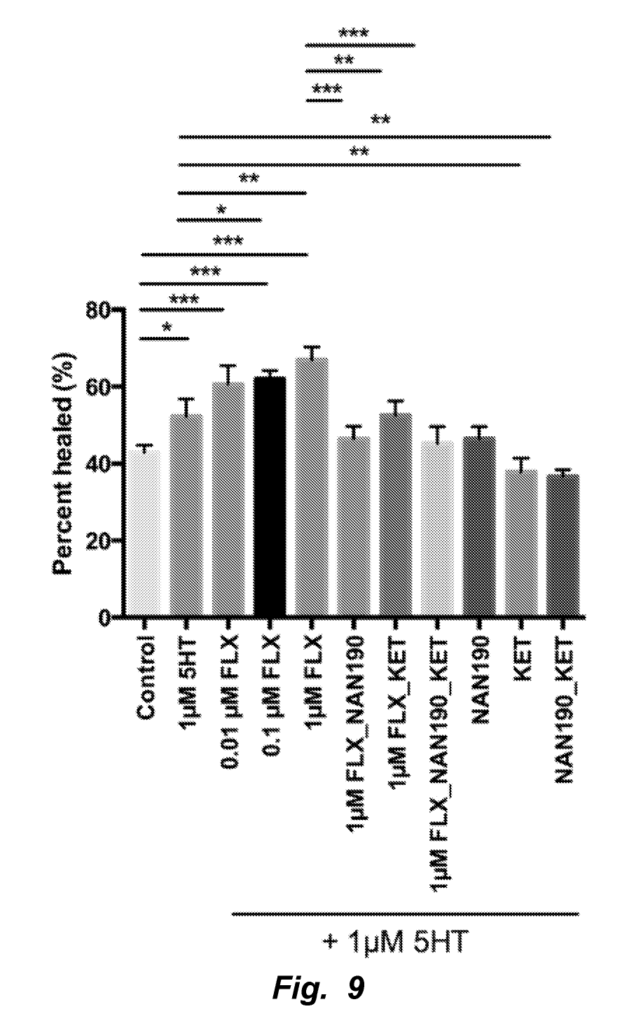

Serotonin receptor expression has previously been found in rodent skin. Furthermore, both the dermis and epidermis contain enzymes capable of transforming tryptophan to serotonin [6, 7]. However, a role for serotonin in cutaneous wound healing has yet to be elucidated. Serotonin was found to enhance migration of keratinocytes in a dose-dependent manner (FIG. 2A, B). Fluoxetine is known to increase serotonin levels by inhibiting re-uptake and destruction of serotonin. In the presence of serotonin, fluoxetine also significantly increased the migratory speed of human keratinocytes (FIG. 9). Moreover, inhibition of 5HT signaling through 5HTR1A and 5HTR2a abrogated this effect. Furthermore, we found that serotonin significantly increased phosphorylation of ERK and STAT3 (FIG. 2C) which has been shown to be critical for keratinocyte migration [8]. We also observed an increase in NF-kB protein (FIG. 2C) that is important in controlling cellular survival and differentiation [9, 10]. Importantly, the effects seem limited to cell migration and not cell proliferation as the increase in scratch wound re-epithelialization was observed in the presence of the cell cycle inhibitor mitomycin. This finding demonstrates keratinocyte migration is a defining terminal event in wound healing and this indicates that the serotonergic pathway, as targeted by fluoxetine, may be beneficial in wound healing.

Topical Fluoxetine and Topical Serotonin Increase Re-Epithelialization in a Diabetic Mouse Model of Impaired Wound Healing.

Given the increase in keratinocyte migration we observed in vitro, we next set out to determine whether or not fluoxetine could aid in re-epithelialization in vivo. Full-thickness excisional biopsy wounds were treated with topical fluoxetine, topical serotonin, or vehicle control. We found that topical treatment with either fluoxetine or serotonin significantly improved re-epithelization (FIG. 4A, B, C, D). This finding demonstrates that this novel topical formulation of fluoxetine improves wound healing in a model that mimics human diabetic non-healing wounds, a costly problem to both patients and the healthcare system.

Topical Fluoxetine and Topical Serotonin Modulate the Local Immune Milieu in Wound Beds.

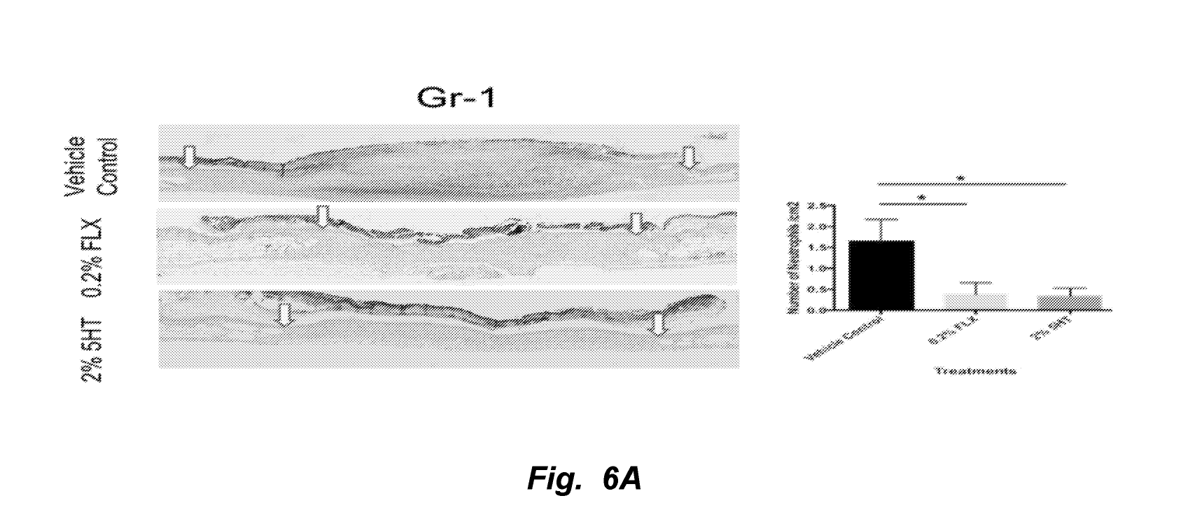

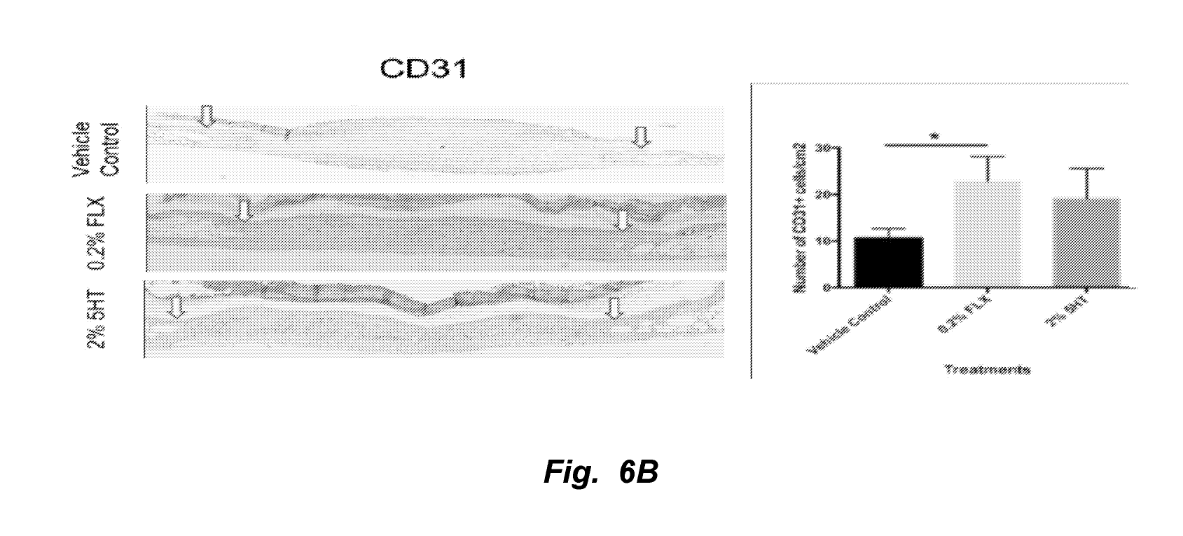

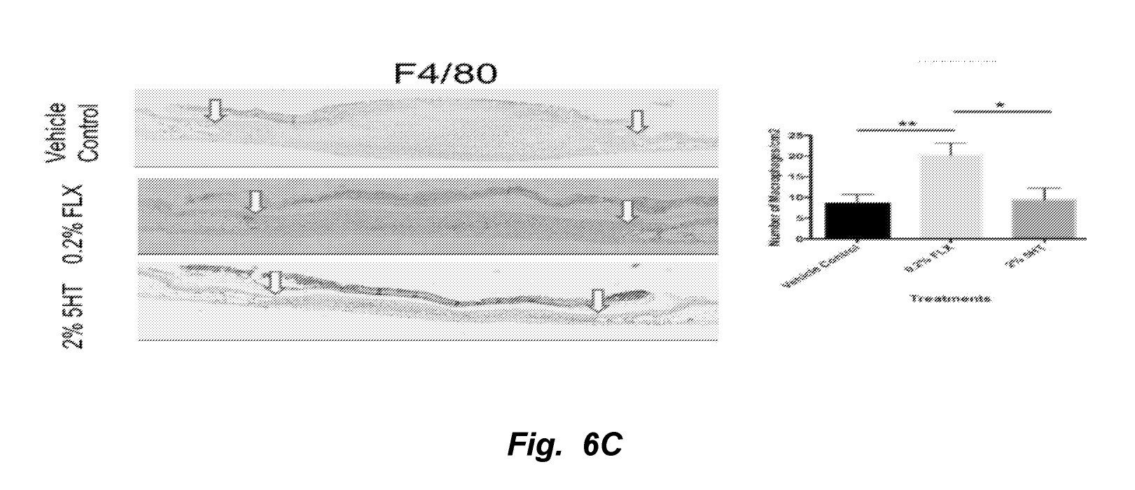

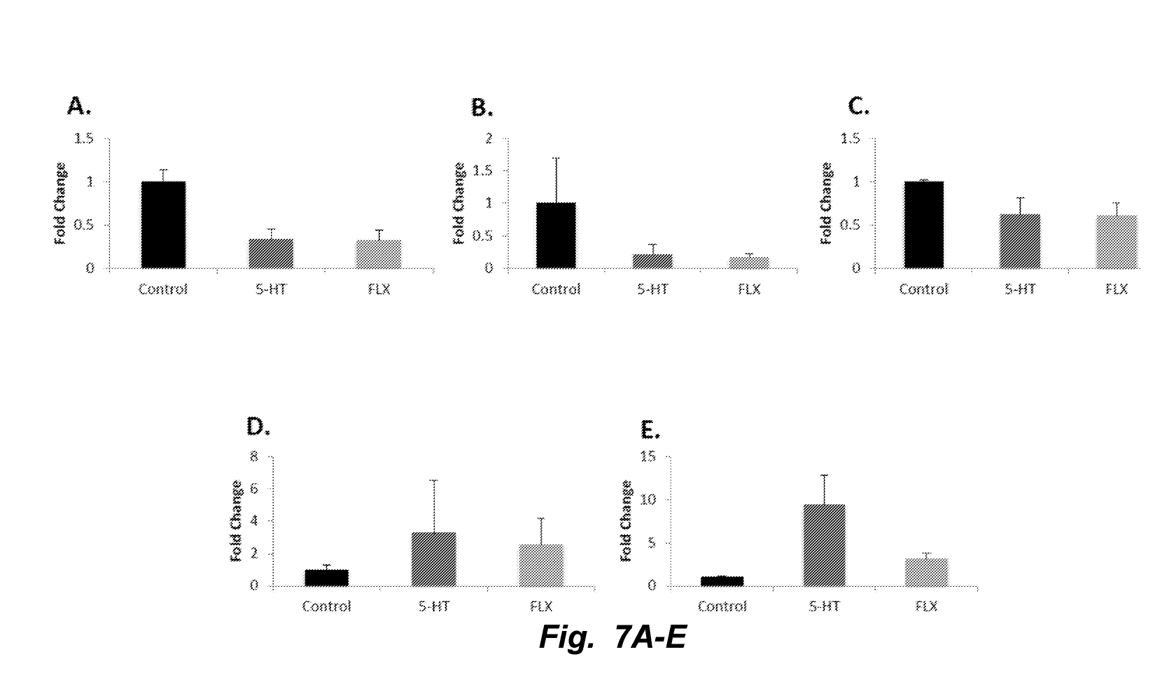

Given the improvement in wound healing that we observed in vivo and the fact that chronic wounds are known to persist in the presence of chronic inflammation, we set out to immunophenotype the wound beds in mice treated with fluoxetine and serotonin compared to vehicle controls. Importantly, significantly fewer neutrophils (Gr-1+ cells), potent pro-inflammatory regulators, were noted in wound beds that had been treated with fluoxetine or serotonin compared to vehicle-treated controls (FIG. 6A). Neutrophil persistence in wound beds is known to contribute to impaired wound healing [11]. Surprisingly, macrophage (F4/80+ cells) numbers were significantly increased in fluoxetine-treated mice (FIG. 6C). Macrophages polarized towards an anti-inflammatory M2 phenotype are known to be key mediators of angiogenesis in wound healing [12]. Along with this potential pro-healing anti-inflammatory shift, angiogenesis (CD31+ endothelial cells) was significantly increased in mice treated with fluoxetine (FIG. 6B). This finding demonstrates the mechanism behind fluoxetine's significant improvement in diabetic wound healing in vivo.

Fluoxetine Enhances Macrophage Polarization Towards an Anti-Inflammatory, Pro-Angiogenic M2 Phenotype.

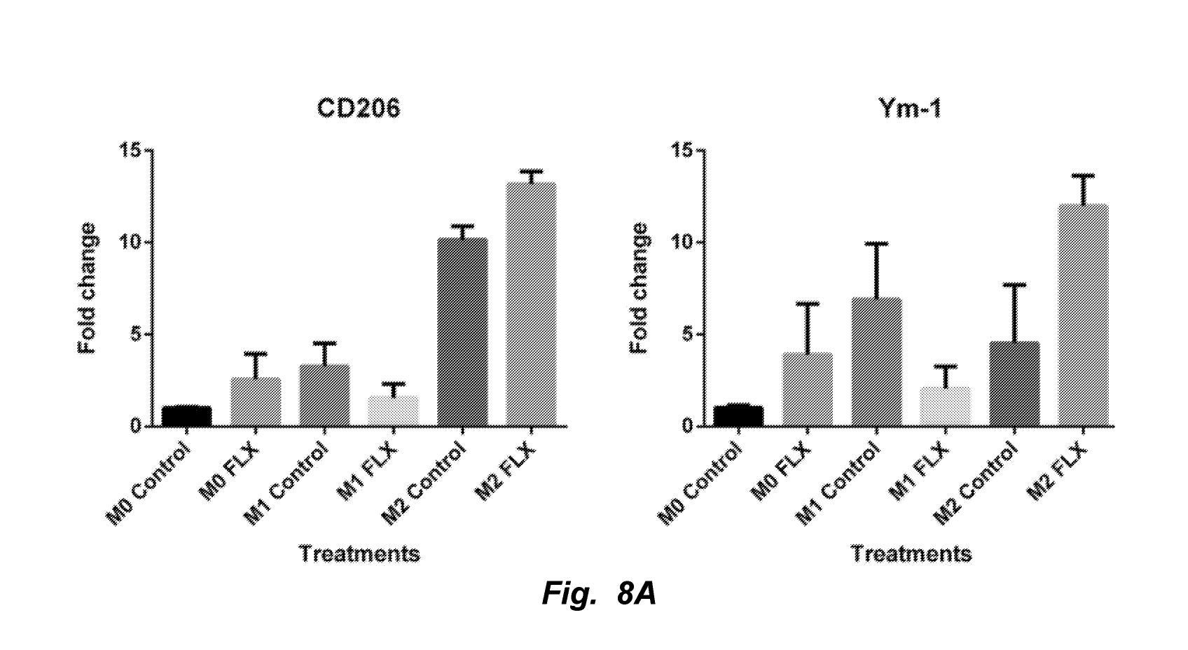

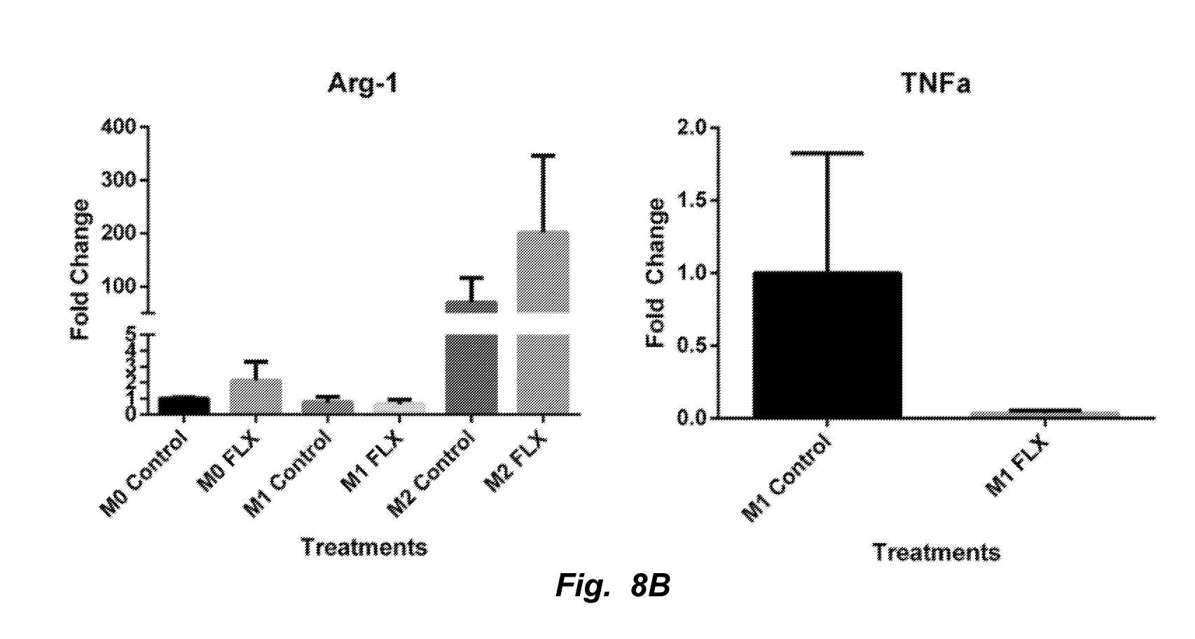

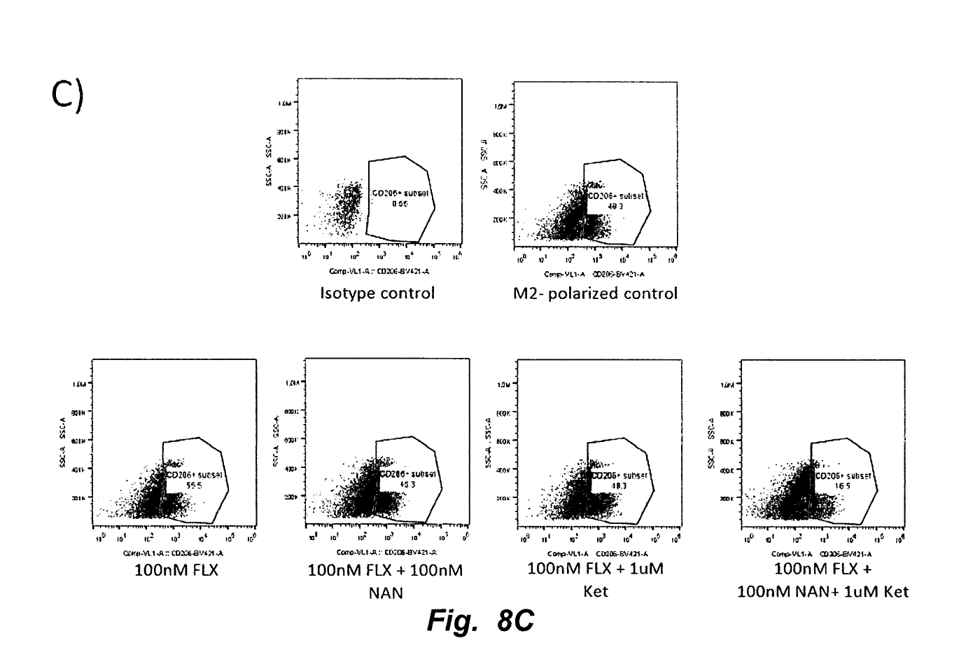

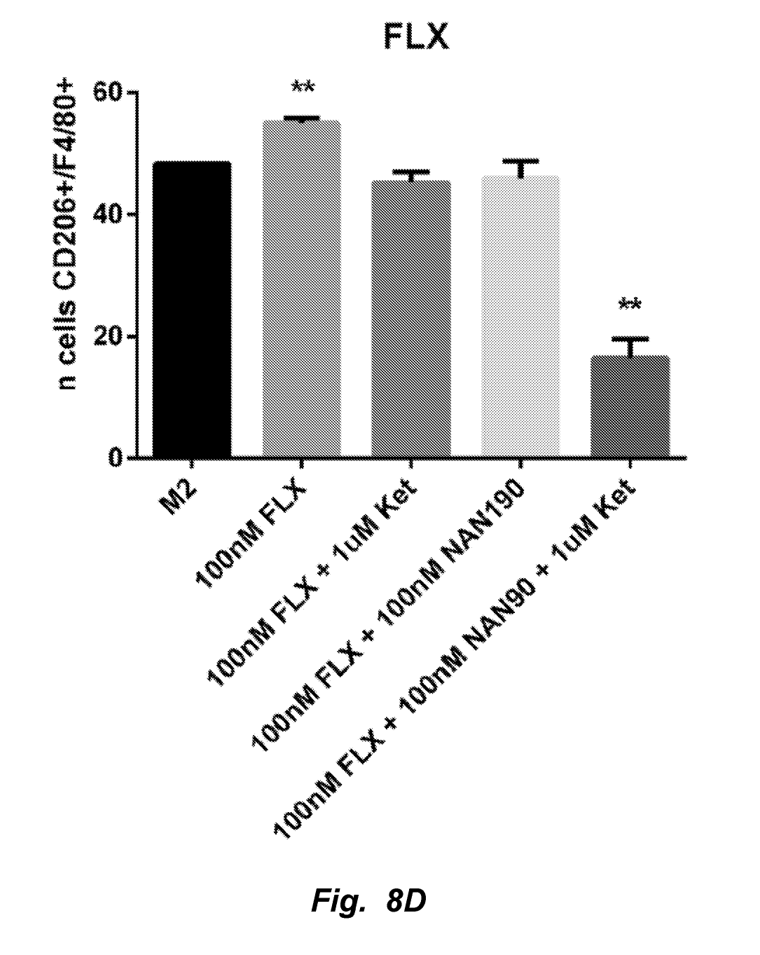

Serotonin is known to modulate polarization of macrophages towards an anti-inflammatory, pro-reparative, M2 phenotype [13]. Given the observation of an increase in both macrophages and angiogenesis in the fluoxetine-treated wound beds, we tested the ability of fluoxetine to influence macrophage polarization. Bone marrow-derived monocytes were successfully matured into macrophages based on F4/80 and CD11b positivity. After 4 hours of polarization, RT-PCR demonstrated that fluoxetine significant increased Arg-1, CD206, and Ym-1 transcripts, indicating that fluoxetine may enhance M2 polarization (FIG. 8A,B). This finding was confirmed by flow cytometry after 24 hours of M2 polarization. M2 polarization of naïve macrophages was enhanced by fluoxetine and this effect was reversed by addition of Ketanserin (5HTR2A inhibitor) and NAN190 (5HTR1A inhibitor) (FIG. 8C,D). Moreover, the combination of both receptor blockers worked synergistically to even further reduce M2 polarization, as indicated by CD206+ cells. This data is consistent with the conclusion that fluoxetine's anti-inflammatory effect is mediated by multiple serotonin receptors.

MSCs (Both Adipose- and Bone Marrow-Derived), Serotonin, and Fluoxetine all Inhibit T Cell Proliferation In Vitro, Independent of 5HTR2A.

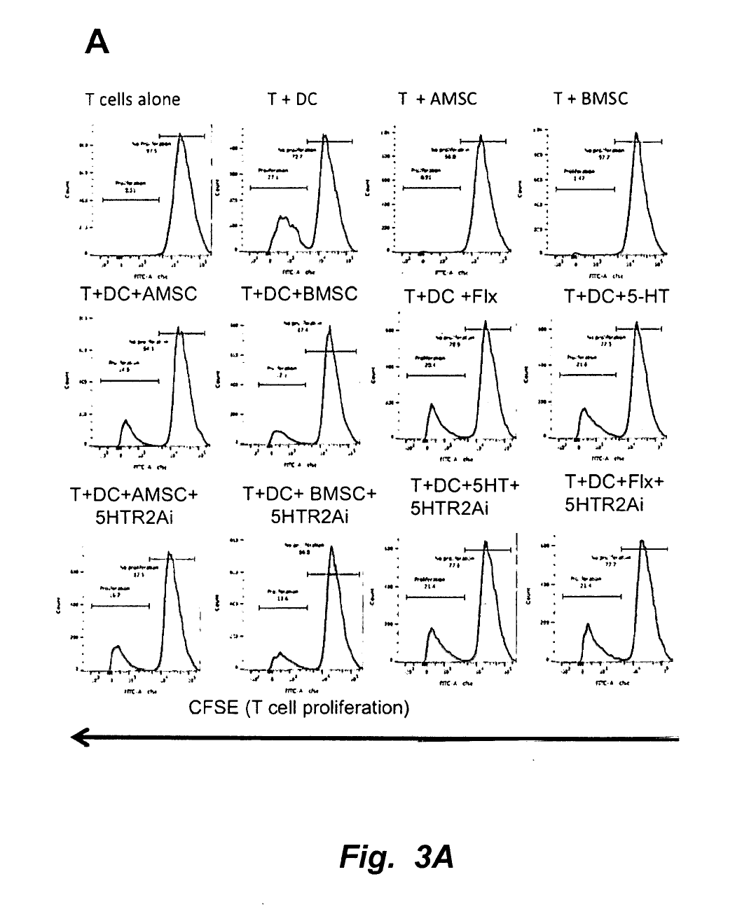

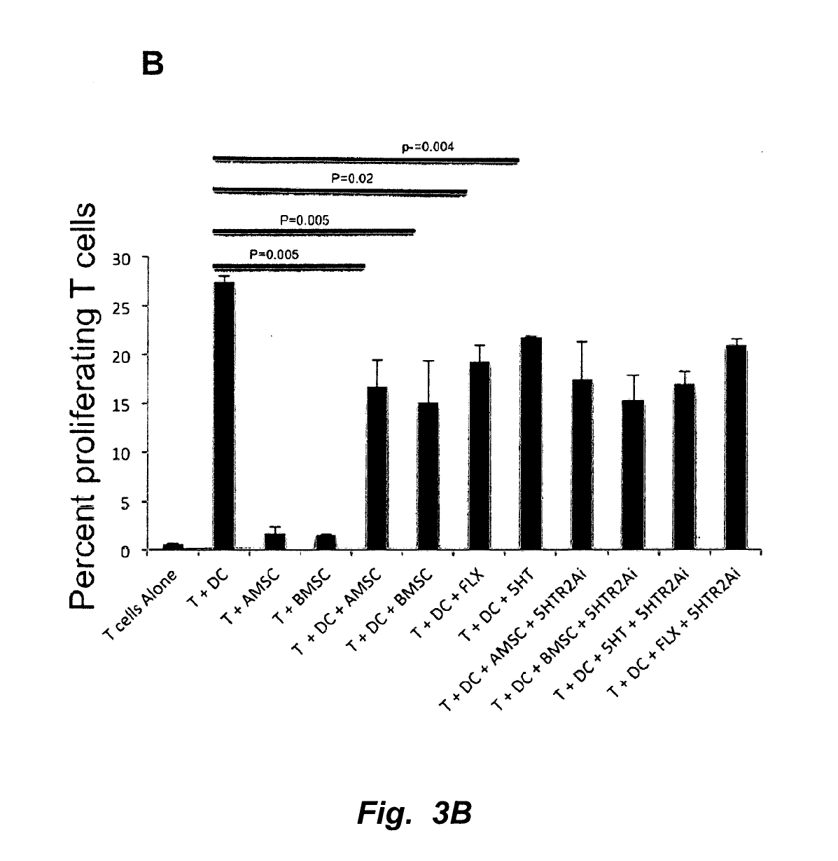

As demonstrated above, fluoxetine demonstrated anti-inflammatory effects in vivo by decreasing neutrophil numbers and increasing M2, anti-inflammatory macrophages. We next determined whether or not this serotonin pathway affected T cells. Using allogeneic dendritic cells to stimulate T cell proliferation, we demonstrated that MSC from either adipose or bone marrow as well as serotonin or fluoxetine were able to significantly inhibit this proliferation (FIG. 3A, B). Importantly, viability was assessed using annexin-V staining and there was no difference between treatment groups. Moreover, this effect appeared to be independent of 5HTR2A, indicating that other 5HT receptors may play a more prominent role in this cell population. This finding elucidates a broader anti-inflammatory role for the serotonergic pathway.

Combinatorial Therapy with Topical Fluoxetine and Topical Timolol Increase Re-Epithelialization in a Diabetic Mouse Model of Impaired Wound Healing.

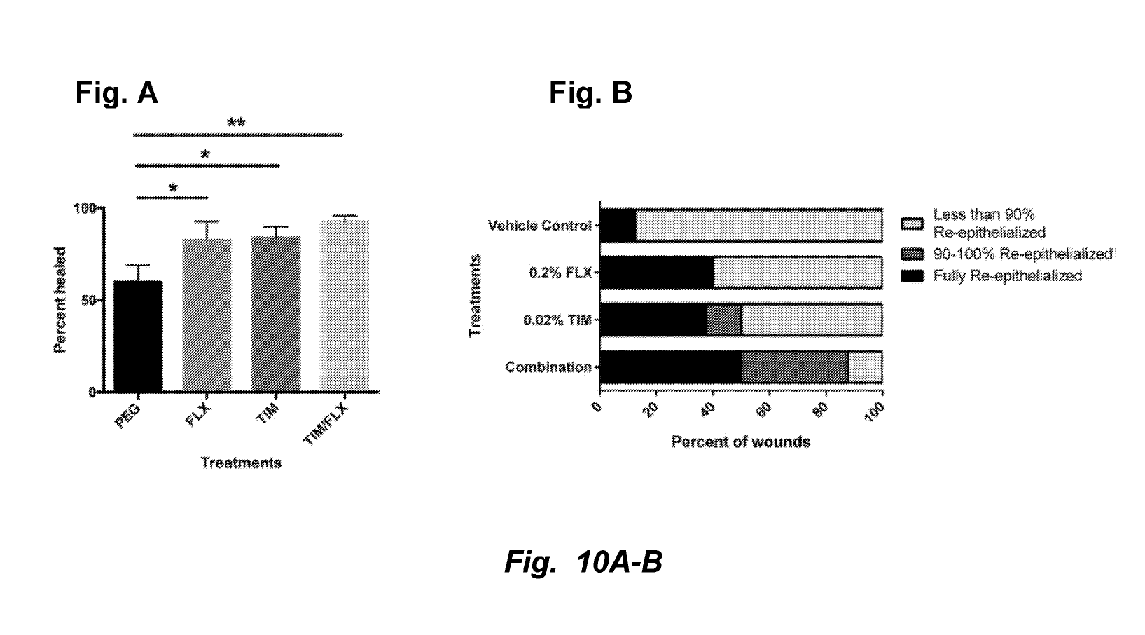

Topical fluoxetine and timolol each individually increased re-epithelialization compared to PEG vehicle control. Surprisingly, combinational therapy with topical fluoxetine/timolol was statistically superior to either treatment alone (FIG. 10A, B). This is significant as it demonstrates that targeting other pathways along with serotonin may be beneficial in healing chronic non-healing wounds, which are often multifactorial in origin. A combined therapeutic approach may derive from this finding.

Combinatorial Therapy with Topical Fluoxetine and BMSC Increases Re-Epithelialization.

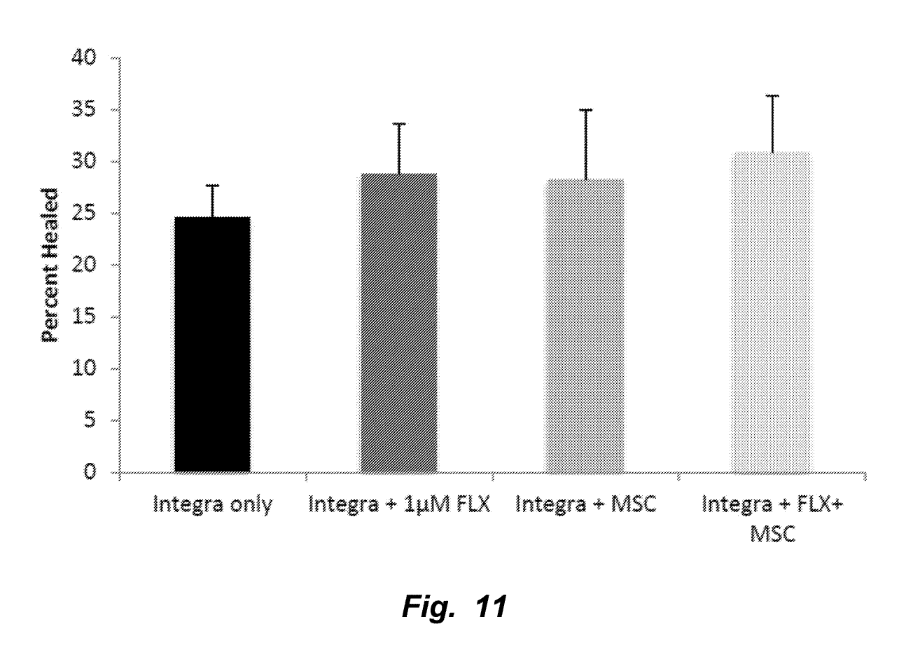

MSC have previously been shown to be beneficial in wound healing. As our in vitro data (described above) demonstrate that fluoxetine increases both MSC migration and keratinocyte migration and proliferation in vitro, we sought to determine whether combinatorial therapy with topical fluoxetine and BMSC improves wound healing in vivo. It is important to note that all wounds in this experiment were seeded with an INTEGRA™ matrix and that low dose fluoxetine was used (1 μM). 1 μM fluoxetine alone increased re-epithelialization by day 11 by 17% compared to INTEGRA™ alone. MSC-seeding of the INTEGRA™ matrix increased re-epithelialization by 13% compared to INTEGRA™ alone. Interestingly, there was a 25% improvement in re-epithelialization when topical fluoxetine was delivered with MSC pre-seeded INTEGRA™ matrices (FIG. 11). This demonstrates that fluoxetine may enhance the pro-healing properties of MSC, and also provides potential for a combinatorial therapy.

Topical delivery of fluoxetine represents a safe therapy with minimum systemic absorption. Finally, because the goal of this work is for eventual translation in to clinical use, we examined the systemic absorption of fluoxetine after topical delivery to a wound bed in the diabetic mouse. We found that after 10 days of daily dosing (0.2% w/v FLX), the levels of fluoxetine and its metabolite (norfluoxetine) in mouse sera ranged from 87-138 ng/ml (Table 1) which is much lower than plasma levels in patients treated with oral fluoxetine at therapeutic doses and is also significantly lower than levels in oral and intraperitoneal injections in mice [14, 15]. This finding indicates the potential of topically-delivered fluoxetine for wound healing with minimal systemic effects compared to other delivery methods (Table 1).

TABLE 1

| | |
|---|---|
| Day 12 | Serum level of FLX (ng/ml) |

| Animal | Vehicle | |
|---|---|---|
| number | Control | 0.2% FLX |
| 1 | not detected | 138.686 ± 39.3 |
| 2 | not detected | 125.009 ± 0.75 |
| 3 | not detected | 87.735 ± 0.70 |
| 4 | not detected | 92.8985 ± 9.01 |
| 5 | not detected | 103.3185 ± 7.28 |

Fluoxetine level in sera of mice treated with topical FLX versus vehicle control. Quantification was done using ELISA. Data represents as mean ± sd.

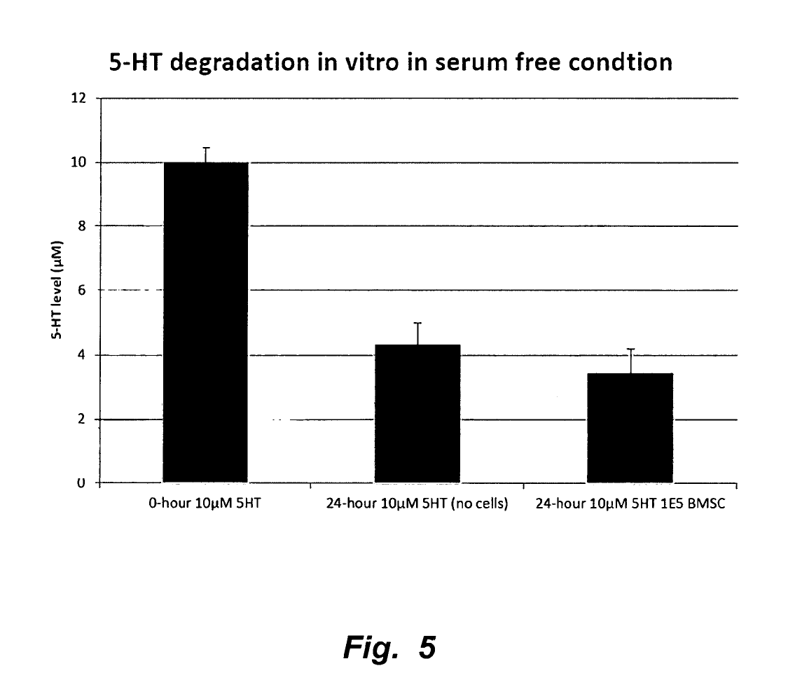

Serotonin is rapidly degraded when exposed to in vitro in culture conditions. Over 50% of serotonin in culture was lost after 24 hours in culture in both the presence and absence of BMSC. Our data show that topical formulation of fluoxetine increases available local extracellular serotonin with a daily dosing regimen, however (FIG. 5).

In conclusion, chronic, non-healing wounds represent a significant source of morbidity with great cost to both patients and the healthcare system in the United States and therapeutic options are currently limited. Herein, we have demonstrated both the pro-epithelial migration and anti-inflammatory properties of the serotonin pathway in wound healing, which we have targeted using a topical formulation of fluoxetine. Using a diabetic mouse model of impaired wound healing, we have demonstrated that topical fluoxetine both improves wound re-epithelialization and targets the local immune milieu, leading to a decrease in the neutrophil infiltration, a more anti-inflammatory phenotype and improved angiogenesis in the wound bed. We also demonstrate the therapeutic utility of a number of combinatorial approaches using fluoxetine combined with other drugs or cells. We believe that the serotonigenic pathway represents a safe and effective target for the challenging clinical problem of chronic, non-healing wounds

### REFERENCES

1. Rheinwald, J. G. and H. Green, Serial cultivation of strains of human epidermal keratinocytes: the formation of keratinizing colonies from single cells. Cell, 1975. 6(3): p. 331-43.
2. Isseroff, R. R., et al., Conversion of linoleic acid into arachidonic acid by cultured murine and human keratinocytes. J Lipid Res, 1987. 28(11): p. 1342-9.
3. Yates, D. T., et al., Technical note: Effects of rumen passage on fluoxetine bioavailability in serum and effects of fluoxetine on serum prolactin concentration and demeanor in ewes1. Journal of Animal Science, 2010. 88(11): p. 3611-3616.
4. Tartar, D. M., et al., FoxP3+RORgammat+ T helper intermediates display suppressive function against autoimmune diabetes. J Immunol, 2010. 184(7): p. 3377-85.
5. Park, S. A., et al., Full-thickness splinted skin wound healing models in db/db and heterozygous mice: implications for wound healing impairment. Wound Repair Regen, 2014. 22(3): p. 368-80.
6. Slominski, A., A. Pisarchik, and J. Wortsman, Expression of genes coding melatonin and serotonin receptors in rodent skin. Biochim Biophys Acta, 2004. 1680(2): p. 67-70.
7. Nordlind, K., E. C. Azmitia, and A. Slominski, The skin as a mirror of the soul: exploring the possible roles of serotonin. Exp Dermatol, 2008. 17(4): p. 301-11.
8. Sano, S., et al., Keratinocyte-specific ablation of Stat3 exhibits impaired skin remodeling, but does not affect skin morphogenesis. Embo j, 1999. 18(17): p. 4657-68.
9. Lippens, S., et al., Keratinocyte-specific ablation of the NF-[kappa]B regulatory protein A20 (TNFAIP3) reveals a role in the control of epidermal homeostasis. Cell Death Differ, 2011. 18(12): p. 1845-1853.
10. Adams, S., et al., Regulation of NF-kappaB activity and keratinocyte differentiation by the RIP4 protein: implications for cutaneous wound repair. J Invest Dermatol, 2007. 127(3): p. 538-44.
11. Kim, M. H., et al., Catecholamine stress alters neutrophil trafficking and impairs wound healing by beta2-adrenergic receptor-mediated upregulation of IL-6. J Invest Dermatol, 2014. 134(3): p. 809-17.
12. Fantin, A., et al., Tissue macrophages act as cellular chaperones for vascular anastomosis downstream of VEGF-mediated endothelial tip cell induction. Blood, 2010. 116(5): p. 829-40.
13. de las Casas-Engel, M., et al., Serotonin skews human macrophage polarization through HTR2B and HTR7. J Immunol, 2013. 190(5): p. 2301-10.
14. Hodes, G. E., et al., Sex-specific effects of chronic fluoxetine treatment on neuroplasticity and pharmacokinetics in mice. J Pharmacol Exp Ther, 2010. 332(1): p. 266-73.
15. Dulawa, S. C., et al., Effects of chronic fluoxetine in animal models of anxiety and depression. Neuropsychopharmacology, 2004. 29(7): p. 1321-30.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A composition comprising as sole active agent an antidepressant in a pharmaceutically acceptable carrier, wherein the composition is formulated for topical delivery of the antidepressant to a tissue or organ.

2. A composition comprising as sole active agents an antidepressant in combination with a beta adrenergic receptor antagonist, both active agents in a pharmaceutically acceptable carrier, wherein the composition is formulated for topical delivery of the antidepressant and the beta adrenergic receptor antagonist to a tissue or organ.

3. The composition of any one of claims 1 to 2, wherein the tissue or organ is other than the eye.

4. The composition of any one of claims 1 to 3, wherein the tissue or organ comprises an epithelial tissue.

5. The composition of any one of claims 1 to 4, wherein the composition is formulated for topical delivery of the antidepressant, optionally in combination with the beta adrenergic receptor antagonist, to skin.

6. The composition of any one of claims 1 to 5, wherein the antidepressant increases extracellular serotonin levels.

7. The composition of any one of claims 1 to 5, wherein the antidepressant is selected from the group consisting of a selective serotonin reuptake inhibitor (SSRI), a serotonin-norepinephrine reuptake inhibitor (SNRI), a tricyclic or tetracyclic antidepressant (TCA), a monoamine oxidase inhibitor (MAOI) and an atypical antidepressant.

8. The composition of any one of claims 1 to 7, wherein the selective serotonin reuptake inhibitor (SSRI) is selected from the group consisting of fluoxetine, citalopram, escitalopram, fluvoxamine, fluvoxamine CR, paroxetine, paroxetine CR, and sertraline.

9. The composition of any one of claims 2 to 8, wherein the beta adrenergic receptor antagonist is a non-selective antagonist for β1 and β2 adrenergic receptors.

10. The composition of claim 9, wherein the beta adrenergic receptor antagonist is selected from carteolol, carvedilol, labetalol, nadolol, penbutolol, pindolol, propranolol, sotalol, timolol, and mixtures, analogs and salts thereof.

11. The composition of any one of claims 2 to 8, wherein the beta adrenergic receptor antagonist is a selective antagonist for β1 adrenergic receptors.

12. The composition of claim 11, wherein the beta adrenergic receptor antagonist is selected from acebutolol, atenolol, betaxolol, bisoprolol, celiprolol, esmolol, metoprolol, nebivolol, and mixtures, analogs and salts thereof.

13. The composition of any one of claims 2 to 8, wherein the beta adrenergic receptor antagonist is a selective antagonist for β2 adrenergic receptors.

14. The composition of claim 13, wherein the β2 adrenergic receptor antagonist is selected from butoxamine and ICI-118,551.

15. The composition of any one of claims 2 to 8, wherein the beta adrenergic receptor antagonist is selected from the group consisting of timolol, labetalol, dilevelol, propanolol, carvedilol, nadolol, carteolol, penbutolol, sotalol, ICI-118,551, butoxamine, and mixtures, analogs and salts thereof.

16. The composition of any one of claims 2 to 15, wherein the beta adrenergic receptor antagonist is substantially free of activity as a beta-3 adrenergic receptor agonist.

17. The composition of any one of claims 1 to 16, comprising the sole active agent or agents at a concentration in the range of about 0.001% w/v to about 30% w/v.

18. The composition of any one of claims 2 to 17, comprising one or both of the antidepressant and the beta adrenergic receptor antagonist in a subtherapeutic dose.

19. The composition of any one of claims 1 to 18, wherein the composition further comprises mesenchymal stem cells (MSCs).

20. The composition of claim 19, wherein the MSCs have been have been contacted and/or pre-conditioned with an antidepressant and/or beta adrenergic receptor antagonist.

21. The composition of any one of claims 19 to 20, wherein the MSCs have been cultured in medium comprising the antidepressant and/or beta adrenergic receptor antagonist.

22. The composition of any one of claims 19 to 21, wherein the MSCs have been cultured at least 24 hours in medium comprising the antidepressant and/or beta adrenergic receptor antagonist.

23. The composition of any one of claims 19 to 22, wherein the MSCs have been cultured under hypoxic conditions.

24. The composition of any one of claims 19 to 23, wherein the MSCs are derived from a tissue selected from the group consisting of adipose, bone marrow, dermis, placenta, umbilical cord, and Wharton's jelly.

25. The composition of any one of claims 19 to 24, wherein the MSCs are human.

26. The composition of any one of claims 1 to 25, wherein the composition comprises a gel, liquid, ointment, cream, lotion, suspension, spray or foam.

27. An extracellular matrix scaffold comprising the composition of any one of claims 19 to 26.

28. The extracellular matrix scaffold of claim 27, wherein the scaffold comprises collagen.

29. A dressing comprising the composition of any one of claims 1 to 26, wherein the dressing is impregnated with the composition or wherein at least one surface of the dressing is coated with the composition.

30. A kit comprising the composition of any one of claims 1 to 26 and/or the extracellular matrix scaffold of any one of claims 27 to 28 and/or the dressing of claim 29, packaged in one or more containers.

31. A method for increasing a rate of wound healing in a subject in need thereof, the method comprising:
a) identifying a subject suffering from a wound in an epithelial tissue; and
b) topically administering the composition of any one of claims 1 to 26 and/or the extracellular matrix scaffold of any one of claims 27 to 28 and/or the dressing of claim 29 to the subject.

32. The method of claim 31, wherein the epithelial tissue comprises skin.

33. The method of any one of claims 31 to 32, wherein a combination of an antidepressant and a beta adrenergic receptor antagonist are topically co-administered and one or both of the antidepressant and the beta adrenergic receptor antagonist are administered at a subtherapeutic dose.

34. The method of any one of claims 31 to 33, wherein the composition or dressing or extracellular matrix scaffold comprises MSCs, and the MSCs are autologous, syngeneic, allogeneic or xenogeneic to the subject.

35. The method of any one of claims 31 to 34, wherein the wound comprises a chronic skin wound.

36. The method of any one of claims 31 to 35, wherein the wound is secondary to vascular insufficiency/injury or a connective tissue disease.

37. The method of any one of claims 31 to 36, wherein the wound comprises a venous stasis ulcer, a diabetic foot ulcer, a peripheral digit ulcer, a neuropathic ulcer, or a decubitus ulcer.

38. The method of any one of claims 31 to 36, wherein the wound comprises a wound resulting from surgical wound dehiscence.

39. The method of any one of claims 31 to 36, wherein the wound comprises an incision, laceration, abrasion, or ulcer.

40. The method of any one of claims 31 to 36, wherein the wound comprises a burn.

41. The method of claim 31, wherein the epithelial tissue comprises a genitourinary epithelium, a gastrointestinal epithelium, a pulmonary epithelium, or a corneal epithelium.

42. The method of any one of claims 31 to 41, wherein the rate of wound healing is at least about 10% greater in comparison to an untreated individual or the same subject prior to topical administration of the composition.

43. The method of any one of claims 31 to 42, wherein the subject is a human.

44. The method of any one of claims 31 to 43, wherein the antidepressant or the combination of the antidepressant and the beta adrenergic receptor antagonist is administered to the subject multiple times.

45. The method of any one of claims 31 to 44, wherein the antidepressant or the combination of the antidepressant and the beta adrenergic receptor antagonist is administered to the subject at least once daily.

46. The method of any one of claims 31 to 45, wherein the antidepressant or the combination of the antidepressant and the beta adrenergic receptor antagonist is administered to the subject at least once daily for at least 10 days.
